(19) United States
(12) Patent Application Publication
(10) Pub. No.: US 2024/0401145 A1
(43) Pub. Date: Dec. 5, 2024

(54) METHOD FOR PREDICTING THE RESPONSE OF A PATIENT DIAGNOSED WITH CANCER TO TREATMENT AND/OR IMAGING WITH A COMPOUND TARGETING CCK2-R, AND COMPOUND FOR USE IN METHODS OF SELECTIVELY TREATING AND/OR IMAGING CANCER

(71) Applicant: Paul Scherrer Institut, Villigen PSI (CH)

(72) Inventors: Anna Pokorska-Bocci, Denens (CH); Antoine Attinger, Colombier (CH)

(21) Appl. No.: 18/696,532

(22) PCT Filed: Sep. 28, 2021
(86) PCT No.: PCT/EP2021/076701

§ 371 Date: Mar. 28, 2024

(51) Int. Cl. C12Q 1/6886; A61K 51/08

(52) U.S. Cl. CPC C12Q 1/6886; A61K 51/088; C12Q 2600/106; C12Q 2600/158

(57) ABSTRACT

A predictive method and a compound for use in treating and/or imaging cancer. The present invention relates to a method for predicting the response of a patient diagnosed with cancer to treatment and/or imaging with a compound targeting cholecystokinin 2 receptor. There is also described a compound targeting CCK2R for use in methods of selectively treating and/or imaging cancer in a patient diagnosed therewith, which leads to improved delivery and therapeutic efficacy and/or enables improved imaging of the tumor tissues.

The present invention relates to a predictive method and a compound for use in methods of treating and/or imaging cancer. In particular, the present invention relates to a method for predicting the response of a patient diagnosed with cancer to treatment and/or imaging with a compound targeting CCK2-R, e.g., a radiolabeled gastrin analogue. Furthermore, the present invention also relates to a compound targeting CCK2-R, e.g., a radiolabeled gastrin analogue, for use in methods of selectively treating and/or imaging cancer in a patient diagnosed therewith, which leads to improved delivery and therapeutic efficacy (antitumor activity) and/or enables improved imaging of the tumor tissues.

### BACKGROUND OF THE INVENTION

G-protein coupled receptors (GPCRs) constitute a superfamily of membrane proteins whose function is to transduce a chemical signal across the cell membrane. GPCRs targeted by agonistic ligands undergo conformational changes, which lead to the exchange of GDP for GTP on the G-protein alpha subunit (Gα). Subsequent dissociation of the Gα and Gβγ subunits from the receptor results in activation of various kinase signaling pathways involving protein kinases A and C (PKA; PKC) as well as phosphoinositide 3-kinase (PI3K) and mitogen activated protein kinases (MAPKs) (O'Hayre et al. Cuff Opin Cell Biol. 2014, 27, 126-135). Subsequently, activated GPCRs undergo desensitization via an arrestin-mediated internalization process, whereby they can be trafficked to lysosomes for degradation, or to endosomes for their recycling back to the cell surface (Rajagopal et al. Cell Signal. 2018, 41, 9-16). This internalization process enables the delivery of ligand-conjugated radioactive nuclides into target cells, e.g., cancer cells.

Overexpression of GPCRs that selectively bind their peptide ligands allow the development of peptide receptor radionuclide therapy (PRRT) for human cancers (Lappano et al. Nat Rev Drug Discov. 2011, 10(1), 47-60). One of the most important goal of PRRT is to achieve high tumor uptake of radiolabeled ligands. Therefore, strategies to increase the uptake of radiopharmaceuticals in tumors or cancer tissues while sparing healthy organs from cytotoxic side effects have been considered.

High expression of cholecystokinin 2 receptor (CCK2-R), which belongs to the GPCR family, has been validated in some particular forms of cancer including, e.g., medullary thyroid cancer (MTC), small-cell lung cancer (SCLC), gastrointestinal stromal tumor (GIST), gliomas, as well as colorectal cancer (CRC), breast cancer (BC), and ovarian cancer (Reubi et al. Cancer Res 1997, 57(7), 1377-1386). Furthermore, the small peptide hormone “minigastrin” is known to bind CCK2-R with high affinity. Previous studies have therefore suggested to use radiolabeled (mini)gastrin-derived peptides for targeted PRRT.

WO 2015/067473 A1 describes the use of gastrin analogues DOTA-(DGln)₆-Ala-Tyr-Gly-Trp-Nle-Asp-Phe-NH₂ (named as “PP-F10N”) and DOTA-(DGlu)₆-Ala-Tyr-Gly-Trp-Nle-Asp-Phe-NH₂ (named as “PP-F11N”) labeled with a radionuclide, such as ¹¹¹In, to target MTC tissues. The gastrin analogues of WO 2015/067473 A1 showed good chemical stability (i.e., good resistance to proteolysis and oxidation) as well as good biodistribution in animal models (i.e., a good uptake in transplanted CCKBR-expressing A431 cells and low accumulation in the kidneys) and hence, they are suited for clinical applications.

Nonetheless, the success of such applications depends on the protein (CCK2-R) expression levels in the targeted tissues. Moreover, the protein expression levels can significantly vary depending on (1) the individual tumor, (2) the patient, and (3) the technology used for detection. In certain indications, the prevalence of CCK2-R in the tumor tissues is low, with only about 10% to 20% of patients showing sufficient expression levels to enable PRRT. The assessment of CCK2-R expression levels by standard methods, such as immunohistochemistry (IHC) or autoradiography (RA), has also proven very difficult. This is because these methods are constraining, require a high level of optimization, and/or do not produce reproducible results, e.g., likely because available anti-CCK2-R antibodies do not exhibit sufficient selectivity and/or specificity.

For these reasons, it can be extremely difficult to predict whether treatment and/or imaging (diagnosis) of tumor tissues with a compound targeting CCK2-R e.g. radiolabeled ligands targeting CCK2-R, in a particular patient will be successful. Patients receiving PRRT, but whose disease does not express CCK2-R (or insufficient levels thereof), may be unnecessarily exposed to radiation, albeit without any clinical benefit. There is hence a need for a robust predictive method that allows to identify those patients who can, or are likely to benefit from treatment and/or imaging with radiolabeled ligands targeting CCK2-R.

In view of the foregoing, it is an object of the present invention to provide a method to predict the response of a patient diagnosed with cancer to treatment and/or imaging with a compound targeting CCK2-R, e.g., a radiolabeled gastrin analogue.

It is a further object of the present invention to provide a compound for use in methods of selectively treating and/or imaging cancer, which achieves sufficient tumor uptake and biodistribution (i.e., tumor-to-healthy tissue ratio) of a CCK2-R ligand, e.g., radiolabeled gastrin analogue, thus leading to excellent therapeutic efficacy and/or imaging of the tumor tissues, while side-effects due to unspecific accumulation of radioactivity in healthy tissues can be prevented.

### SUMMARY OF THE PRESENT INVENTION

The present invention provides a method to predict the response of a patient diagnosed with cancer to treatment and/or imaging with a compound targeting CCK2-R, e.g., a radiolabeled gastrin analogue. The present inventors have found that the mRNA expression levels of CCKBR in tumor cells closely correlate with the protein (CCK2-R) expression levels and also with the binding of radiolabeled ligands to CCK2-R, so that the CCKBR mRNA expression level constitutes a robust predictive biomarker of the clinical response to treatment and/or imaging with a compound targeting CCK2-R e.g. radiolabeled ligands targeting CCK2-R. The correlation between mRNA and protein expression levels is particularly surprising, because the mRNA expression level is known to be a very poor indicator of the expression of the protein products (for a discussion on the poor correlation between mRNA and protein expression, see, e.g., Koussounadis et al. Scientific Reports 2015, 5, 10775; Wang D. Comput Biol Chem. 2008, 32(6), 462-468). In particular for CCK2-R, the association between mRNA and protein expression levels is known to be very weak and statistically not significant (Mjønes et al. Horm Canc 2018, 9, 40-54).

The present invention thus relates to a method for predicting the response of a patient diagnosed with cancer to treatment and/or imaging with a compound targeting CCK2-R comprising the steps of:
(a) assaying a tumor sample from a patient diagnosed with cancer for the mRNA expression level of CCKBR; and
(b) determining whether the mRNA expression level of CCKBR is equal to, or greater than a predetermined cut-off range (or value) to predict if the patient is likely to respond to treatment and/or imaging with a compound targeting CCK2-R;
   wherein the compound targeting CCK2-R is preferably a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a radionuclide, preferably DOTA or NODAGA, more preferably DOTA.

Furthermore, the present invention also relates to a compound for use in a method of selectively treating and/or imaging cancer in a patient diagnosed therewith comprising the steps of:
(a) assaying a tumor sample from a patient diagnosed with cancer for the mRNA expression level of CCKBR;
(b) determining whether the mRNA expression level of CCKBR is equal to, or greater than a predetermined cut-off range (or value) to predict if the patient is likely to respond to treatment and/or imaging with a compound targeting CCK-2R; and
(c) selectively administering an effective dose of a compound targeting CCK-2R to the patient;
   wherein the compound targeting CCK2-R is preferably a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a radionuclide, preferably DOTA or NODAGA, more preferably DOTA.

In one aspect, the present invention relates to a compound for use in a method of selectively treating cancer in a patient diagnosed therewith comprising the steps of:
(a) assaying a tumor sample from a patient diagnosed with cancer for the mRNA expression level of CCKBR;
(b) determining whether the mRNA expression level of CCKBR is equal to, or greater than a predetermined cut-off range to predict if the patient is likely to respond to treatment with a compound targeting CCK2-R;
(c) selectively administering an imaging dose of a first compound targeting CCK2-R to the patient to obtain an image of body parts or tissues; and (d) selectively administering a treatment dose of a second compound targeting CCK2-R to the patient;
   wherein the first compound targeting CCK2-R is preferably a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a first radionuclide, preferably DOTA or NODAGA, more preferably DOTA; and
wherein the second compound targeting CCK2-R is preferably a radiolabeled gastrin analogue represented by formula (1), wherein X represents a moiety that chelates a second radionuclide, preferably DOTA or NODAGA, more preferably DOTA.

The present invention in particular includes the following embodiments (“Items”):

1. Method for predicting the response of a patient diagnosed with cancer to treatment and/or imaging with a compound targeting CCK2-R comprising the steps of:
(a) assaying a tumor sample from a patient diagnosed with cancer for the mRNA expression level of cholecystokinin B receptor (CCKBR); and
(b) determining whether the mRNA expression level of CCKBR is equal to, or greater than a predetermined cut-off range to predict if the patient is likely to respond to treatment and/or imaging with a compound targeting CCK2-R;
   wherein the compound targeting CCK2-R is preferably a radiolabeled gastrin analogue represented by formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a radionuclide, preferably DOTA or NODAGA, more preferably DOTA.

2. The method according to item 1, wherein the compound targeting CCK2-R is a gastrin analogue represented by formula (1), and the radionuclide is selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm, preferably from ¹⁷⁷Lu, ⁹⁰Y, ⁶⁸Ga, ²²⁵Ac and ¹¹¹In, more preferably from ¹⁷⁷Lu and ⁶⁸Ga.

3. The method according to item 1 or 2, wherein the patient is diagnosed with a disease selected from medullary thyroid cancer (MTC), gliomas, small-cell lung cancer (SCLC), extrapulmonary small-cell carcinoma (EPSCC), gastroenteropancreatic neuroendocrine tumors (GEP-NET), gastrointestinal stromal tumors (GIST), non-small cell lung cancer (NSCLC), colorectal cancer (CRC), astrocytomas, stomach cancer, ovarian cancer, breast cancer (BC), and any other disease expressing the cholecystokinin 2 receptor (CCK2-R).

4. The method according to any one of items 1 to 3, wherein the patient is diagnosed with a disease selected from SCLC, GIST, CRC, BC and NSCLC, preferably from SCLC and GIST, more preferably with SCLC.

5. The method according to any one of items 1 to 4, wherein the tumor sample is a biopsy sample, such as a paraffin-embedded and fixed biopsy sample, a fresh biopsy sample, a frozen biopsy sample, or a sample derived from a core needle or fine-needle aspiration biopsy.

6. The method according to any one of items 1 to 5, wherein the mRNA expression level of CCKBR is determined by reverse transcription-polymerase chain reaction (RT-PCR), RNA sequencing, or any other method for assaying mRNA expression levels in cells, preferably by RT-PCR or RNA sequencing, more preferably by RNA-sequencing.

7. The method according to any one of items 1 to 6, wherein the cut-off range is from 0.4 to 2.0 log 2 transcripts per million (TPM), preferably from 0.5 to 1.8 log 2 TPM, more preferably from 0.6 to 1.4 log 2 TPM, such as 0.6 to 1.0 log 2 TPM.

8. Compound for use in a method of selectively treating and/or imaging cancer in a patient diagnosed therewith comprising the steps of:
(a) assaying a tumor sample from a patient diagnosed with cancer for the mRNA expression level of CCKBR;
(b) determining whether the mRNA expression level of CCKBR is equal to, or greater than a predetermined cut-off range to predict if the patient is likely to respond to treatment and/or imaging with a compound targeting CCK2-R; and
(c) selectively administering an effective dose of compound targeting CCK2-R to the patient;
   wherein the compound targeting CCK2-R is preferably a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a radionuclide, preferably DOTA or NODAGA, more preferably DOTA.

9. The compound for use according to item 8, wherein the compound targeting CCK2-R is a gastrin analogue represented by formula (1), and the radionuclide is selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm, preferably from ¹⁷⁷Lu, ⁹⁰Y, ⁶⁸Ga, ²²⁵Ac and ¹¹¹In, more preferably from ¹⁷⁷Lu and ⁶⁸Ga.

10. The compound for use according to item 8 or 9, wherein the patient is diagnosed with a disease selected from MTC, gliomas, SCLC, EPSCC, GEP-NET, GIST, NSCLC, CRC, astrocytomas, stomach cancer, ovarian cancer, BC, and any other disease expressing CCK2-R.

11. The compound for use according to any one of items 8 to 10, wherein the patient is diagnosed with a disease selected from SCLC, GIST, CRC, BC and NSCLC, preferably from SCLC and GIST, more preferably with SCLC.

12. The compound for use according to any one of items 8 to 11, wherein the tumor sample is a biopsy sample, such as a paraffin-embedded and fixed biopsy sample, a fresh biopsy sample, a frozen biopsy sample, or a sample derived from a core needle or fine-needle aspiration biopsy.

13. The compound for use according to any one of items 8 to 12, wherein the mRNA expression level of CCKBR is determined by RT-PCR, RNA sequencing, or any other method for assaying mRNA expression levels in cells, preferably by RT-PCR or RNA sequencing, more preferably by RNA-sequencing.

14. The compound for use according to any one of items 8 to 13, wherein the cut-off range is from 0.4 to 2.0 log 2 TPM, preferably from 0.5 to 1.8 log 2 TPM, more preferably from 0.6 to 1.4 log 2 TPM, such as 0.6 to 1.0 log 2 TPM.

15. The compound for use according to any one of items 8 to 14, wherein the compound targeting CCK2-R is a radiolabeled gastrin analogue represented by formula (1) wherein X chelates ¹⁷⁷Lu, and the effective dose of the compound administered to the patient is preferably selected from:
(i) a treatment dose of from 1.0 to 15.0 GBq, preferably from 2.0 to 12.0 Gbq, more preferably from 5.0 to 10.0 GBq, in particular from 6.0 to 8.0 GBq, such as about 6.5 GBq; and
(ii) an imaging dose of from 0.5 to 3.0 GBq, preferably from 0.7 to 2.5 GBq, more preferably from 1.0 to 2.0 GBq, such as about 1.85 GBq.

16. The compound for use according to any one of items 8 to 14, wherein compound targeting CCK2-R is a radiolabeled gastrin analogue represented by formula (1) wherein X chelates ⁶⁸Ga, and the effective dose of the compound administered to the patient is preferably from 0.5 to 4 MBq/Kg/person, preferably from 1 to 3 MBq/Kg/person, such as about 2 MBq/Kg/person.

17. The compound for use according to any one of items 8 to 16, wherein the compound is administered to the patient once or twice per cycle of two to ten weeks, preferably once per cycle of four to eight weeks, more preferably once per cycle of six weeks or once per cycle of eight weeks.

18. The compound for use according to any one of items 8 to 17, wherein the compound is administered to the patient
(A) once per cycle of six weeks for at least two six-week cycles, preferably once per cycle of six weeks for at least three six-week cycles, more preferably once per cycle of six weeks for three six-week cycles; or
(B) once per cycle of eight weeks for at least two eight-week cycles, preferably once per cycle of eight weeks for at least three eight-week cycles, more preferably once per cycle of eight weeks for three eight-week cycles.

19. The compound for use according to any one of items 8 to 18, wherein the compound is administered concurrently with, before and/or after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, or any other targeted therapies.

20. Compound for use in a method of selectively treating cancer in a patient diagnosed therewith comprising the steps of:
(a) assaying a tumor sample from a patient diagnosed with cancer for the mRNA expression level of CCKBR;
(b) determining whether the mRNA expression level of CCKBR is equal to, or greater than a predetermined cut-off range to predict if the patient is likely to respond to treatment with a compound targeting CCK2-R;
(c) selectively administering an imaging dose of a first compound targeting CCK2-R to the patient to obtain an image of body parts or tissues; and
(d) selectively administering a treatment dose of a second compound targeting CCK2-R to the patient;
   wherein the first compound targeting CCK2-R is preferably a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a first radionuclide, preferably DOTA or NODAGA, more preferably DOTA; and
wherein the second compound targeting CCK2-R is preferably a radiolabeled gastrin analogue represented by formula (1), wherein X represents a moiety that chelates a second radionuclide, preferably DOTA or NODAGA, more preferably DOTA.

21. The compound for use according to item 20, wherein each of the first and second compounds targeting CCK2-R is a radiolabeled gastrin analogue represented by formula (1), and wherein each of the first and second radionuclides is independently selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm, preferably from ¹⁷⁷Lu, ⁹⁰Y, ⁶⁸Ga, ²²⁵Ac and ¹¹¹In, and more preferably from ¹⁷⁷Lu and ⁶⁸Ga.

22. The compound for use according to item 20 or 21, wherein each of the first and second compounds targeting CCK2-R is a radiolabeled gastrin analogue represented by formula (1), and wherein,
the first radionuclide is ⁶⁸Ga, and the imaging dose administered to the patient is preferably from 0.5 to 4 MBq/Kg/person, more preferably from 1 to 3 MBq/Kg/person, such as about 2 MBq/Kg/person; and/or
the second radionuclide is ¹⁷⁷Lu, and the treatment dose administered to the patient is preferably from 1.0 to 15.0 GBq, more preferably from 2.0 to 12.0 Gbq, even more preferably from 5.0 to 10.0 GBq, in particular from 6.0 to 8.0 GBq, such as about 6.5 GBq.

23. The compound for use according to any one of items 20 to 22, wherein the patient is diagnosed with a disease selected from MTC, gliomas, SCLC, EPSCC, GEP-NET, GIST, NSCLC, CRC, astrocytomas, stomach cancer, ovarian cancer, BC, and any other disease expressing CCK2-R.

24. The compound for use according to any one of items 20 to 23, wherein the patient is diagnosed with a disease selected from SCLC, GIST, CRC, BC and NSCLC, preferably from SCLC and GIST, more preferably with SCLC.

25. The compound for use according to any one of items 20 to 24, wherein the tumor sample is a biopsy sample, such as a paraffin-embedded and fixed biopsy sample, a fresh biopsy sample, a frozen biopsy sample, or a sample derived from a core needle or fine-needle aspiration biopsy.

26. The compound for use according to any one of items 20 to 25, wherein the mRNA expression level of CCKBR is determined by RT-PCR, RNA sequencing, or any other method for assaying mRNA expression levels in cells, preferably by RT-PCR or RNA sequencing, more preferably by RNA-sequencing.

27. The compound for use according to any one of items 20 to 26, wherein the cut-off range is 0.4 to 2.0 log 2 TPM, preferably 0.5 to 1.8 log 2 TPM, more preferably 0.6 to 1.4 log 2 TPM, such as 0.6 to 1.0 log 2 TPM.

28. The compound for use according to any one of items 20 to 27, wherein the first and/or second compound(s) is/are administered concurrently with, before and/or after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, or any other targeted therapies.

### FIGURE

FIG. 1—Graphical representation showing the correlation between the binding of ¹¹¹In-PP-F111N to CCK2R (x-axis) and the mRNA expression level of CCKBR (y-axis) in biological samples (i.e., tumor, patient derived xenograft, healthy samples) from patients diagnosed with cancer, i.e., GIST, SCLC, NSCLC, MTC.

### DETAILED DESCRIPTION OF THE PRESENT INVENTION

### 1. Definitions

The expression “gastrin analogue” as used herein refers to a class of compounds (peptides) structurally related to the endogenous peptide hormone gastrin, which can bind to CCK2-R. Gastrin is a linear peptide hormone produced by G cells of the duodenum and in the pyloric antrum of the stomach. It is secreted into the bloodstream. The encoded polypeptide is pre-progastrin, which is cleaved by enzymes in posttranslational modification to produce progastrin and then gastrin in various forms, including primarily big-gastrin (G-34), little gastrin (G-17), and minigastrin. CCK is a peptide hormone structurally related to gastrin in that both compounds share five C-terminal amino acids. CCK exists naturally in several forms including, e.g., CCK8. Gastrin and peptide hormones related thereto typically contain the same C-terminal amino acid motif, which enables their binding to CCK2-R.

The pharmacological activity of a given gastrin analogue towards CCK2-R can be determined by measuring the intracellular increase of calcitonin level in gastrin analogue-stimulated cells as described by Blaker et al. (Regulatory Peptides 2004, 118, 111-117).

The gastrin analogue used in the present invention is preferably represented by the following formula (1):

X-(DGlu)₆-Ala-Tyr-Gly-Trp-Nle-Asp-Phe-NH₂ (1)

Wherein X represents a moiety that chelates a radionuclide, such as DOTA or NODAGA, wherein DOTA or NODAGA chelates a radionuclide, such as ¹⁷⁷Lu or ⁶⁸Ga.

“DOTA” refers to the chelating moiety 1,4,7,10-tetraazacyclododecane-1,4,7,10-tetraacetic acid, which is covalently attached to the N-terminus of the peptide chain via one of its carboxyl group. The compound of formula (1) wherein X is DOTA corresponds to the compound named as “PP-F11N”.

“NODAGA” refers to the chelating moiety 1,4,7-triazacyclononane,1-glutaric acid-4,7-acetic acid, which is covalently attached to the N-terminus of the peptide chain via one of its carboxyl group.

Unless specified otherwise or dictated otherwise by the context, all connections between adjacent amino acid groups are formed by peptide (amide) bonds. The peptides described herein are listed in the conventional amino- to carboxy-direction from left to right.

A “compound targeting CCK2-R” (or “compound capable of targeting CCK2-R”), refers to a compound that can bind to CCK2-R, such as CCK2-R agonists, e.g., (mini-)gastrin and derivatives thereof, non-sulfated gastrin, CCK, non-sulfated CCK, RB-400, and PBC-264, as well as CCK2-R antagonists (for examples of suitable CCK2-R antagonists, see Kaloudi et al. Mol Pharm. 2020, 17(8), 3116-3128). The gastrin analogue of formula (1) is to be understood as compound targeting CCK2-R.

The expression “moiety that chelates a radionuclide” as used herein refers to a moiety, such as DOTA, which can donate electrons to a radionuclide to form a coordination complex therewith, i.e., by forming at least one coordinate covalent (dipolar) bond therewith. The chelating mechanism depends on the chelating agent and/or radionuclide. For example, it is believed that DOTA can coordinate a radionuclide via carboxylate and amino groups (donor groups) thereby forming complexes having high stability (Dai et al. Nature Com. 2018, 9, 857). Non-limiting examples of moieties that can chelate a radionuclide (all suitable for use moiety “X” in the compound of formula (1)) include diethylenetriamine pentaacetic acid (DTPA), cyclohexyl diethylenetriamine pentaacetic acid (CH-X-DTPA), desferrioxamine (DFO), N1-(27-amino-11,22-dihydroxy-7,10,18,21-tetraoxo-6,11,17,22-tetraazaheptacosyl)-N1-hydroxy-N4-(5-(N-hydroxyacetamido)pentyl)succinimide (DFO′), N1-(5-(3-(4-aminobutyl)-1-hydroxy-2-oxopiperidine-3-carboxamido)pentyl)-N1-hydroxy-N4-(5-(N-hydroxy-4-((5-(N-hydroxyacetamido)pentyl)amino)-4-oxobutanamido)pentyl)succinimide (DFO-cyclo′), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), 1,4,7-trazacyclononane-1,4,7-triacetic acid (NOTA), mercaptoacetyl-glycyl-glycyl-glycine (maGGG), mercaptoacetyl-serine-serine-serine (maSSS), 1,4,7,10-tetraatacyclododecane-1,4,7,10-tetraacetic acid-methonine (DOTA-Met), ethylenediaminetetraacetic acid (EDTA), ethylenediaminediacetic acid, triethylenetetraminehexaacetic acid (TTHA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)tiacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-Tetraazabicyclo[9.3.1]pentadeca-1(15),11,13-triene-3,6,9-triacetic acid (PCTA), 2,2′,2″,2′″-(1,4,7,10-tetraazacyclotrdecane-1,4,7,10-tetrayl)tetraacetic acid (TRITA), 2,2′,2″,2′″-(1,4,7,10-tetraazacyclotrdecane-1,4,7,10-tetrayl)tetraacetamide (TRITAM), 2,2′,2″-(1,4,7,10-tetraazacyclotridecane-1,4,7-triyl)tracetamide (TRITRAM), trans-N-dimethyl-cyclam, 2,2′,2″-(1,4,7-triazacyclononane-1,4,7-triyl)tracetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA).

The term “cancer” as used herein means the pathological condition in mammalian tissues that is characterized by abnormal cell growth to form malignant tumors, which may have the potential to invade or spread to other tissues or parts of the body to form “secondary” tumors known as metastases. A tumor comprises one or more cancer cells.

The expression “patient diagnosed with cancer” as used herein refers to a human patient having a positive diagnosis with respect to at least one type of cancerous disease. In one aspect, the patient has been positively diagnosed with at least one type of cancer known to express CCK2-R, such as medullary thyroid cancer (MTC), gliomas, small-cell lung cancer (SCLC), extrapulmonary small-cell carcinoma (EPSCC), gastroenteropancreatic neuroendocrine tumors (GEP-NET), gastrointestinal stromal tumors (GIST), non-small cell lung cancer (NSCLC), colorectal cancer (CRC), astrocytomas, stomach cancer, ovarian cancer, and breast cancer. A “positive diagnosis” means that the patient has a histological and/or cytological status of disease and, optionally, one or more of the following:
(1) radiographically documented disease progression or recurrence after at least one systemic treatment regimen;
(2) at least one non-irradiated extracranial measurable target lesion as per the Response Evaluation Criteria in Solid Tumors 1.1 (RECIST 1.1) documented after the last anticancer therapy;
(3) at least one tumor lesion as per Positron Emission Tomography Response Criteria in Solid Tumors 1.0 (PERCIST 1.0); and
(4) an Eastern Cooperative Oncology Group (ECOG) score of 0-2.

Preferably, a positive diagnosis of cancer means that the patient has one or more of the following (i) to (viii):
(i) a serum creatinine of less than or equal to 1.5×ULN or an estimated glomerular filtration rate (eGFR) greater than 50 mL/min based on the Chronic Kidney Disease-Epidemiology Collaboration (CKD-EPI) equation;
(ii) a blood absolute neutrophil count (ANC) equal to or greater than 1,500 cells/μL;
(iii) a blood platelet count equal to or greater than 100,000 cells/μL;
(iv) a blood haemoglobin equal to or greater than 9 g/dL;
(v) AST or ALT equal to or less than 3'ULN;
(vi) a serum albumin greater than 20 g/L;
(vii) a total bilirubin equal to or less than 2.5 mg/dL; and
(viii) an alkaline phosphatase less than 5×ULN.

The expression “tumor uptake” (of radiopharmaceuticals) refers to the biological process in which molecules, e.g., a radiolabeled gastrin analogue, are taken up by tumor (cancer) cells. Tumor uptake includes tumor cell uptake of molecules and/or their retention in the tumor microenvironment. As a result, the molecules can be present inside the tumor (cancer) cell, at the cell membrane, e.g., accumulated on the cell membrane, and/or within the tumor microenvironment. The accumulation of radioactivity will then damage tumor cell DNA via direct activity by creating DNA single or double strand brakes, or via indirect activity with the generation of free radicals leading to tumor cell death (Desouky et al. Journal of Radiation Research and Applied Sciences 2015, 8(2), 247-254).

The term “predicting” or “predict” as used herein means that the method provides information to enable a physician to determine whether an individual patient diagnosed with cancer is likely to have a particular clinical response, i.e., a positive (beneficial) or negative response, to treatment and/or imaging with a radiolabeled compound, e.g., a radiolabeled gastrin analogue. It does not refer to the ability to predict whether the patient will respond to treatment and/or imaging with 100% accuracy. Instead, the prediction refers to an increased probability that is more than speculation, but less than certainty.

The expression “predicting the response of the patient to treatment and/or imaging” refers to the ability to predict whether a patient diagnosed with cancer is likely to have a particular clinical response i.e., a positive or negative response, to treatment and/or imaging with a radiolabeled compound. A positive (beneficial) clinical response can refer to any clinical benefit to the patient with regard to treatment and/or diagnosis, including, without limitation, (1) inhibition, at least to some extent, of tumor growth, including slowing down and compete growth arrest, (2) reduction in the number of tumor cells, (3) reduction in tumor size, (4) inhibition, i.e., reduction, slowing down or complete stopping, of tumor cell infiltration into adjacent peripheral organs and/or tissues, (5) inhibition of metastasis, (6) enhancement of anti-tumor immune response, (7) relief, at least to some extent, of one or more symptoms associated with cancer, (8) increase in the length of survival, (9) decreased mortality, and/or (10) visualization of the tumor tissues. A positive response of the patient can also be considered in the context of the individual patient relative to a group of patients having a comparable clinical diagnosis and can include, without limitation, (11) an increase in the duration of Recurrence-Free Interval, (12) an increase in the time of survival as compared to Overall Survival in a population, (13) an increase in the time of Disease-Free Survival, and/or (14) an increase in the duration of Distant Recurrence-Free Interval.

Furthermore, a positive clinical response in the context of “imaging” can refer to the ability (of a physician) to establish a diagnosis of disease in a patient. If a diagnosis is already established by another method, imaging can be used to confirm the first diagnosis, establish a second diagnosis, monitoring the state and/or progression of disease, or the like. Imaging can be performed by administering an imaging dose of a (radiolabeled) compound targeting CCK2-R, e.g., a radiolabeled gastrin analogue, to a patient, and subsequently visualizing the tracer by a suitable method, such as SPECT or PET. In one aspect, imaging is used to (i) collect data, (ii) comparing the data with standard values, (iii) finding any significant deviation, e.g., a symptom, during the comparison, and (iv) attributing that deviation to a particular clinical picture to establish diagnosis.

The term “assaying” as used herein refers to the act of identifying, screening, probing, testing, measuring, quantifying or determining. In one aspect, the term “assaying” refers to the act of quantifying a particular genetic biomarker, such as mRNA. Non-limiting examples of methods for assaying the expression level of a genetic biomarker in a biological (tumor) sample include quantitative polymerase chain reaction (PCR), quantitative reverse transcription polymerase chain reaction (RT-PCR), enzyme-linked immunosorbent assay (ELISA), magnetic immunoassay (MIA), flow cytometry, as well as molecular profiling (sequencing) technologies, such as mRNA sequencing platforms which enable sequencing of the whole transcriptome. In one aspect of the present invention, the mRNA expression levels of CKKBR are assayed by RNA sequencing of the whole transcriptome as available from, e.g., Caris Life Sciences®.

The term “tumor sample” as used herein refers to a sample of the cancer with which the patient has been diagnosed, such as a biopsy sample. The tumor sample may be used for the purpose of diagnosis, prediction, and/or monitoring.

The term “expression level” as applied to a gene refers to the normalized level of a gene product, e.g., the normalized value determined for the mRNA expression level of CCKBR. The mRNA expression level as applied to CCKBR can be quantified by the aforementioned methods. The expression data used herein are normalized, which means that the mRNA expression levels are corrected for differences in the amount of RNA assayed and variability in the quality of the RNA used. The assays can provide for normalization by incorporating the expression of certain normalizing genes, which are relatively invariant under the relevant conditions, such as housekeeping genes.

The expression “mRNA of CCKBR” as used herein refers to any mRNA transcription product (transcript) of the gene encoding for the CCK2-R protein (CCK2-R preferably having a sequence as disclosed in GenBank Accession #NP_795344.1). In one aspect, the mRNA expression level corresponds to the expression level of one or more transcripts selected from the sequences disclosed in GenBank Accession #NM_001363552.2, NM_176875.4, NM_001318029.2, and XM_017018516.1, preferably to the expression level of the transcript having the sequence disclosed in GenBank Accession #NM_176875.4). In this connection, it should be noted that the term “CCKBR” (or “cckbr”) refers to the mRNA, whereas the term “CCK2-R” refers to the protein in accordance with the terminology commonly used in the art.

The term “cut-off value” as used herein refers to a predetermined value used for prediction purposes. In particular, when the mRNA expression level in a biological (tumor) sample is equal to or greater than the cut-off value, it predicts that the patient is “CCK2-R positive” and therefore likely to respond to treatment and/or imaging with a compound targeting CCK2-R, e.g., a radiolabeled gastrin analogue, and when the mRNA expression level is below the cut-off value it predicts that the patient is not likely (or less likely) to respond to treatment and/or imaging with a compound targeting CCK2-R, e.g., a radiolabeled gastrin analogue.

Likewise, the term “cut-off range” as used herein refers to a predetermined range used for prediction purposes. In particular, when the mRNA expression level in a biological sample is equal to (i.e., falls within), or greater than the cut-off range, it predicts that the patient is “CCK2-R positive” and therefore likely to respond to treatment and/or imaging with a compound targeting CCK2-R, e.g., a radiolabeled gastrin analogue, and when the mRNA expression level is below the cut-off range, it predicts that the patient is not likely (or less likely) to respond to treatment and/or imaging with a compound targeting CCK2-R, e.g., a radiolabeled gastrin analogue. An expression level that is “equal to a range” is to be understood as meaning that the expression level falls within that range (including the values defining the limits of the range).

The cut-off range (or value) used herein for prediction purposes can be determined by statistical analysis, e.g., Chi-2 statistical analysis, of the mRNA expression levels of CCKBR (as measured by mRNA sequencing) and specific binding of gastrin analogue (i.e., ¹¹¹In-PP-F11N; as measured by autoradiography; see Reubi et al. Cancer Res 1997, 57(7), 1377-1386) in tumor biopsies, healthy tissues (stomach, lung, kidney), and patient derived xenograft (PDX) samples obtained from a panel of patients having an established, positive diagnosis of a disease known to express CCK2-R, such as GIST or SCLC. The cut-off range for distinguishing a sample that is “CCK2-R positive” from other (negative) samples as measured by RA is from 50% to 65% of specific binding of the radiolabeled compound ¹¹¹In-PP-F11N. The cut-off value is 50% as described by Reubi et al. (Cancer Res 1997, 57(7), 1377-1386). The measurements are performed in duplicates. The cut-off range (or value) is normalized and expressed in log 2 of the transcripts per million (TPM).

The term “administering” as used herein refers to the delivery of a compound to a patient by any route.

The term “selectively” as used herein in reference to administering a compound to a patient with cancer means that a particular patient is specifically chosen (selected) from a larger group of patients based on a predetermined criterion, i.e., the likelihood of the patient to respond to treatment and/or imaging with a particular compound e.g. radiolabeled compound. Thus, selective administration differs from standard administration, in which a compound is administered to all patients, regardless of their genetic expression status.

The expression “effective dose” (or “effective amount”) as used herein may refer to the total dose of radioactivity (in Becquerels) administered to a patient in one administration cycle to perform treatment and/or imaging of the tumor tissues, e.g., reducing or stopping cancer cell proliferation, reducing the number of proliferating cancer cells, etc. In one aspect, the effective dose is a “treatment dose” (that may refer to the total dose of radioactivity administered to the patient in one treatment cycle) or an “Imaging dose” (that may refer to the total dose of radioactivity administered to the patient to carry out imaging, such as PET or SPECT/CT imaging, of the tumor tissues). The effective dose is to be understood as the amount of compound targeting CCK2-R, e.g., radiolabeled gastrin analogue of formula (1) alone. Thus, for instance, if a radiolabeled gastrin analogue is administered in combination with another, different radiotherapy the effective dose refers only to the dose of radiolabeled gastrin analogue.

The effective dose can be determined by a physician based on dosimetry. The effective dose and frequency of dosage for any particular subject/patient can vary and depends on a variety of factors including the patient's age, body weight, general health, sex, diet, mode and time of administration, rate of excretion, the severity of the disease, and the individual undergoing therapy. These factors are considered by the physician when determining the effective dose.

The term “about” in relation to a numerical value X means “X±error margin” according to the rounding-off convention applied in the scientific and technical literature: the last decimal place of a numerical indicates its degree of accuracy. For instance, for a dose of 6.5 Gbq, the error margin is 6.45-6.54 GBq.

The term “cycle” as used herein refers to a period of time wherein the compound is administered to the patient (treatment time) and then the patient is allowed to rest (rest time) before entering another cycle. The treatment can include one or more cycles, e.g. up to ten cycles. A series of cycles is usually called a “course”, which can last over several months, e.g. 3 to 6 months, depending on the length of each cycle.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless the context dictates otherwise and/or alternative meanings are explicitly provided herein, all terms are intended to have meanings generally accepted in the art, as reflected by IUPAC Gold Book (status of 1 Nov. 2017), or the Dictionary of Chemistry, Oxford, 6^{th} Ed.

### 2. Overview

The present invention is based on the discovery that the mRNA expression level of CCKBR in tumor tissues of patients diagnosed with cancer closely correlate with the CCK2-R-protein expression levels and also with the specific binding of compounds targeting CCK2-R, e.g., radiolabeled ligands to CCK2-R, so that the mRNA expression level of CCKBR constitutes a robust predictive biomarker of the clinical response to treatment and/or imaging with compounds targeting CCK2-R, e.g., radiolabeled ligands targeting CCK2-R, such as ¹⁷⁷Lu-PP-F11N.

Hence, the mRNA expression level of CCKBR enables to select the patients who can or are likely to respond to treatment and/or imaging with compounds targeting CCK2-R, such as radiolabeled gastrin analogues, e.g. while avoiding unnecessary exposition of other patients e.g. to radiation. This finding is particularly surprising, because mRNA and protein expressions are known to poorly correlate. A prediction based mRNA expression (if at all possible) usually requires the measurement of multiple biomarkers rather than a single biomarker (see Koussounadis et al. Scientific Reports 2015, 5, 10775; Wang D. Comput Biol Chem. 2008, 32(6), 462-468). In particular for CCK2-R, the association between mRNA and protein expression levels is known to be very weak and statistically not significant (Mjønes et al. Horm Canc 2018, 9, 40-54).

One of the most important goal for efficient PRRT is a high tumor uptake of the radiopharmaceutical(s), which depends on the expression level of the targeted receptor, e.g., CCK2-R. It is expected that the methods of the present invention allow to select the patients whose tumor tissues show the required expression levels of CCK2-R to accomplish PRRT, i.e., a high uptake of the radiopharmaceutical, such as a radiolabeled gastrin analogue, in the targeted cancer cells but not in healthy tissues and/or organs, resulting in excellent biodistribution (i.e., tumor-to-healthy tissue ratio) and therapeutic efficacy (i.e., treatment and/or imaging of the tumor tissues), while side-effects due to unspecific accumulation of radioactivity in healthy tissues or organs can be prevented.

### 3. Method for Predicting Response to Treatment and/or Imaging of Disease

The method of the present invention can predict the response of a patient diagnosed with cancer to treatment and/or imaging with a compound targeting CCK2-R, e.g., a radiolabeled gastrin analogue, by analyzing the mRNA expression level of CCKBR in a tumor sample obtained from the patient. The method enables to select the patients who are likely to respond to (benefit from) treatment/imaging with a compound targeting CCK2-R, e.g., a radiolabeled gastrin analogue, and maximize efficacy, while minimizing side effects and avoiding unnecessary exposition of other patients to radiation.

The method comprises the steps of:
(a) assaying a tumor sample from a patient diagnosed with cancer for the mRNA expression level of CCKBR; and
(b) determining whether the mRNA expression level of CCKBR is equal to, or greater than a predetermined cut-off range to predict if the patient is likely to respond to treatment and/or imaging with a compound targeting CCK2-R, e.g., a radiolabeled gastrin analogue.

Any tumor sample taken from a patient diagnosed with a proliferative disease can be used and assayed for the mRNA expression level of CCKBR. In one embodiment, the patient is diagnosed with a disease selected from medullary thyroid cancer (MTC), gliomas, small-cell lung cancer (SCLC), extrapulmonary small-cell carcinoma (EPSCC), gastroenteropancreatic neuroendocrine tumors (GEP-NET), gastrointestinal stromal tumors (GIST), non-small cell lung cancer (NSCLC), colorectal cancer (CRC), astrocytomas, stomach cancer, ovarian cancer, breast cancer (BC), and any other disease expressing CCK2R.

In one embodiment, the patient is diagnosed with a disease selected from SCLC, GIST, CRC, BC and NSCLC, preferably from SCLC and GIST. More preferably, the patient is diagnosed with SCLC.

The sample can be obtained by biopsy or surgical resection. In one embodiment, the tumor sample is a biopsy sample, such as a paraffin-embedded and fixed (archival) sample, a fresh sample, a frozen sample, or a sample derived from a core needle or fine-needle aspiration biopsy. Preferably, the tumor sample is a core needle or fine-needle aspiration biopsy. Once a tumor sample has been obtained, it can be measured directly for its mRNA expression level of CCKBR, or optionally processed beforehand to extract, enrich, and/or isolate the mRNA contained therein by methods known in the art. Such methods are well-known in the art. For instance, methods for mRNA extraction are disclosed, e.g., in standard textbooks of molecular biology, such as Current Protocols of Molecular Biology, Ed. John Wiley & Sons, 1997. Furthermore, mRNA isolation can be performed by using a commercial purification kit, buffer set and protease as available from various manufacturers, such as Qiagen.

In one embodiment, the patient diagnosed with cancer satisfies one or more of the following criteria (i) to (viii) prior to obtaining the tumor sample:
(i) a serum creatinine of less than or equal to 1.5×ULN or an estimated glomerular filtration rate (eGFR) greater than 50 mL/min based on the Chronic Kidney Disease-Epidemiology Collaboration (CKD-EPI) equation;
(ii) a blood absolute neutrophil count (ANC) equal to or greater than 1,500 cells/μL;
(iii) a blood platelet count equal to or greater than 100,000 cells/μL;
(iv) a blood haemoglobin equal to or greater than 9 g/dL;
(v) AST or ALT equal to or less than 3×ULN;
(vi) a serum albumin greater than 20 g/L;
(vii) a total bilirubin equal to or less than 2.5 mg/dL; and
(viii) an alkaline phosphatase less than 5×ULN.

In one embodiment, the method can include a preliminary step of determining whether a patient meets one or more of the above criteria (i) to (viii).

The mRNA expression level of CCKBR can be assayed by using any method known in the art as suitable for assaying mRNA expression levels in cells. Non-limiting examples of suitable methods include nucleic acid sequencing-based methods (mRNA sequencing), reverse transcription-polymerase chain reaction (RT-PCR), microarrays, etc. In one embodiment, the mRNA expression level of CCKBR is assayed by RT-PCR or RNA sequencing. Preferably, the mRNA expression level of CCKBR is assayed by RNA sequencing. This technology is advantageous in that it enables sequencing of the whole transcriptome, allowing analysis of not only coding sequences but also non-coding sequences. Suitable sequencing platforms are commercially available, e.g., from Caris Life Sciences®.

The compound targeting CCK2-R can be any compound capable to bind to CCK2-R, such as a CCK2-R agonist, or a CCK2-R antagonist. In one embodiment, the compound targeting CCK2-R is labeled with a radionuclide (radiolabeled). Preferably, the compound is a radiolabeled gastrin analogue that is represented by the following formula (1):

X-DGlu-DGlu-DGlu-DGlu-DGlu-DGlu-Ala-Tyr-Gly-Trp-Nle-Asp-Phe-NH₂ (1)

wherein X represents a moiety that chelates a radionuclide.

In a preferred embodiment, X represents DOTA or NODAGA (wherein DOTA or NODAGA chelates a radionuclide). More preferably, X is DOTA.

In one embodiment, the compound targeting CCK2-R is a gastrin analogue represented by formula (1), wherein the radionuclide is selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm. In one preferred embodiment, the radionuclide is selected from ¹⁷⁷Lu, ⁹⁰Y, ⁶⁸Ga, ²²⁵Ac and ¹¹¹In, more preferably from ¹⁷⁷Lu and ⁶⁸Ga.

The mRNA expression level of CCKBR assayed in step (a) is used in step (b) to determine whether the patient is likely to respond to treatment and/or imaging with the compound targeting CCK2-R described above. In this respect, the value of the mRNA expression level is compared against a predetermined cut-off range, whereby an mRNA expression level equal to (within) or greater than the cut-off range predicts that the patient is CCK2-R positive and hence likely to respond to treatment and/or imaging with the compound targeting CCK2-R, and an mRNA expression level lower than the cut-off range predicts that the patient is not likely (or less likely) to respond to treatment and/or imaging with the compound targeting CCK2-R.

In one embodiment, the cut-off range is from 0.4 to 2.0 log 2 transcripts per million (TPM), such as 0.4 to 1.0, 0.5 to 1.1, 0.6 to 1.2, 0.7 to 1.3, 0.8 to 1.4, 0.9 to 1.5, 1.0 to 1.6, 1.1 to 1.7, 1.2 to 1.8, 1.3 to 1.9, 1.4 to 2.0, 0.4 to 0.9, 0.5 to 1.0, 0.6 to 1.1, 0.7 to 1.2, 0.8 to 1.3, 0.9 to 1.4, 1.0 to 1.5, 1.1 to 1.6, 1.2 to 1.7, 1.3 to 1.8, 1.4 to 1.9, 1.5 to 2.0, 0.4 to 0.8, 0.5 to 0.9, 0.6 to 1.0, 0.7 to 1.1, 0.8 to 1.2, 0.9 to 1.3, 1.0 to 1.4, 1.1 to 1.5, 1.2 to 1.6, 1.3 to 1.7, 1.4 to 1.8, 1.5 to 1.9, 1.6 to 2.0, 0.4 to 0.7, 0.5 to 0.8, 0.6 to 0.9, 0.7 to 1.0, 0.8 to 1.1, 0.9 to 1.2, 1.0 to 1.3, 1.1 to 1.4, 1.2 to 1.5, 1.3 to 1.6, 1.4 to 1.7, 1.5 to 1.8, 1.6 to 1.9, 1.7 to 2.0, 0.4 to 0.6, 0.5 to 0.7, 0.6 to 0.8, 0.7 to 0.9, 0.8 to 1.0, 0.9 to 1.1, 1.0 to 1.2, 1.1 to 1.3, 1.2 to 1.4, 1.3 to 1.5, 1.4 to 1.6, 1.5 to 1.7, 1.6 to 1.8, 1.7 to 1.9, 1.8 to 2.0, 0.4 to 0.5, 0.5 to 0.6, 0.6 to 0.7, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, or 1.9 to 2.0 log 2 TPM.

In a preferred embodiment, the cut-off range is from 0.5 to 1.8 log 2 TPM, such as 0.5 to 1.1, 0.6 to 1.2, 0.7 to 1.3, 0.8 to 1.4, 0.9 to 1.5, 1.0 to 1.6, 1.1 to 1.7, 1.2 to 1.8, 0.5 to 1.0, 0.6 to 1.1, 0.7 to 1.2, 0.8 to 1.3, 0.9 to 1.4, 1.0 to 1.5, 1.1 to 1.6, 1.2 to 1.7, 1.3 to 1.8, 0.5 to 0.9, 0.6 to 1.0, 0.7 to 1.1, 0.8 to 1.2, 0.9 to 1.3, 1.0 to 1.4, 1.1 to 1.5, 1.2 to 1.6, 1.3 to 1.7, 1.4 to 1.8, 0.5 to 0.8, 0.6 to 0.9, 0.7 to 1.0, 0.8 to 1.1, 0.9 to 1.2, 1.0 to 1.3, 1.1 to 1.4, 1.2 to 1.5, 1.3 to 1.6, 1.4 to 1.7, 1.5 to 1.8, 0.5 to 0.7, 0.6 to 0.8, 0.7 to 0.9, 0.8 to 1.0, 0.9 to 1.1, 1.0 to 1.2, 1.1 to 1.3, 1.2 to 1.4, 1.3 to 1.5, 1.4 to 1.6, 1.5 to 1.7, 1.6 to 1.8, 0.5 to 0.6, 0.6 to 0.7, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, or 1.7 to 1.8 log 2 TPM.

In a more preferred embodiment, the cut-off range is from 0.6 to 1.4 log 2 TPM, such as 0.6 to 1.2, 0.7 to 1.3, 0.8 to 1.4, 0.6 to 1.1, 0.7 to 1.2, 0.8 to 1.3, 0.9 to 1.4, 0.6 to 1.0, 0.7 to 1.1, 0.8 to 1.2, 0.9 to 1.3, 1.0 to 1.4, 0.6 to 0.9, 0.7 to 1.0, 0.8 to 1.1, 0.9 to 1.2, 1.0 to 1.3, 1.1 to 1.4, 0.6 to 0.8, 0.7 to 0.9, 0.8 to 1.0, 0.9 to 1.1, 1.0 to 1.2, 1.1 to 1.3, 1.2 to 1.4, 0.6 to 0.7, 0.8 to 0.9, 0.9 to 1.0, 1.0 to 1.1, 1.1 to 1.2, 1.2 to 1.3, or 1.3 to 1.4 log 2 TPM.

In an even more preferred embodiment, the cut-off range is from 0.6 to 1.0 log 2 TPM, such as 0.6 to 0.8, 0.7 to 0.9, 0.8 to 1.0, 0.6 to 0.7, 0.8 to 0.9, or 0.9 to 1.0 log 2 TPM.

In another embodiment, the value of the mRNA expression level is compared against a predetermined cut-off value, whereby an mRNA expression level equal to or greater than the cut-off value predicts that the patient is likely to respond to treatment and/or imaging with the compound targeting CCK2-R, and an mRNA expression level lower than the cut-off value predicts that the patient is not likely (or less likely) to respond to treatment and/or imaging with the compound targeting CCK2-R. The cut-off value may be selected from the group consisting of 0.4, 0.5, 0.6, 0.7, 0.8, 0.9, 1.0, 1.1, 1.2, 1.3, 1.4, 1.5, 1.6, 1.7, 1.8, 1.9 and 2.0 log 2 TPM, preferably of 0.5, 0.6, 0.7, 0.8, 0.9, 1.0, 1.1, 1.2, 1.3, 1.4 and 1.5 log 2 TPM, more preferably of 0.5, 0.6, 0.7, 0.8, 0.9, 1.0, 1.1 and 1.2 log 2 TPM, and even more preferably of 0.6, 0.7, 0.8, 0.9, 1.0 and 1.1 log 2 TPM.

### 4. Compound for Use in Methods of Treating and/or Imaging Disease

The compound can be used in methods of selectively treating and/or imaging cancer in a patient diagnosed therewith, whereby cancer or tumor cells are treated and/or visualized. The treatment can be a therapeutic and/or a prophylactic treatment, with the aim being to prevent, reduce or stop the progression of the disease via targeted destruction of tumor cells. Imaging (e.g., diagnosis) can be performed by known computer tomography techniques, such as Positron Emission Tomography (PET); for a review of this technique and its application see, e.g., Shankar Vallabhajosula (ed.), Molecular Imaging, Radiopharmaceuticals for PET and SPECT, Springer Verlag or Lucia Martiniova et al., Gallium-68 in Medical Imaging, Current Radiopharmaceuticals, 2016, 9, 187-207.

According to an embodiment, the method of selectively treating and/or imaging cancer comprises the steps of:
(a) assaying a tumor sample from a patient diagnosed with cancer for the mRNA expression level of CCKBR;
(b) determining whether the mRNA expression level of CCKBR is equal to, or greater than a predetermined cut-off range (or value) to predict if the patient is likely to respond to treatment and/or imaging with a compound targeting CCK2-R; and
(c) selectively administering an effective dose of compound targeting CCK2-R to the patient.

In accordance with this embodiment, the steps (a) and (b), and the features defined therein (i.e., tumor sample, mRNA expression level assay, patient diagnosis, cut-off range/value), are as defined above with respect to the method for predicting the response to treatment and/or imaging with a compound targeting CCK2-R.

The compound targeting CCK2-R can be any compound capable to bind to CCK2-R, such as a CCK2-R agonist, or a CCK2-R antagonist. In one embodiment, the compound targeting CCK2-R is radiolabeled. Preferably, the compound targeting CCK2-R is a radiolabeled gastrin analogue represented by the following formula (1):

X-DGlu-DGlu-DGlu-DGlu-DGlu-DGlu-Ala-Tyr-Gly-Trp-Nle-Asp-Phe-NH₂ (1)
wherein X represents a moiety that chelates a radionuclide.

In one embodiment, the compound targeting CCK2-R is a gastrin analogue represented by formula (1), and the radionuclide is selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm. In one preferred embodiment, the radionuclide is selected from ¹⁷⁷Lu, ⁹⁰Y, ⁶⁸Ga, ²²⁵Ac and ¹¹¹In, more preferably from ¹⁷⁷Lu and ⁶⁸Ga.

In one preferred embodiment, the method pertains to treatment of cancer using a compound targeting CCK2-R, and preferably a radiolabeled gastrin analogue of formula (1), wherein DOTA or NODAGA, preferably DOTA, chelates ¹⁷⁷Lu. In another preferred embodiment, the method pertains to imaging of cancer or tumor cells using a compound targeting CCK2-R, and preferably a radiolabeled gastrin analogue of formula (1), wherein DOTA or NODAGA, preferably DOTA, chelates ⁶⁸Ga.

The compound is selectively administered in step (c) to a patient who has been identified in step (b) as CCK2-R positive, i.e. as likely to respond to treatment and/or imaging with a compound targeting CCK2-R, based on the mRNA expression level of CCKBR assayed in step (a). Therefore, the mRNA expression level of CCKBR in the tumor sample is used in step (b) to select those patients that are expected to benefit from treatment and/or imaging with the compound. This is very beneficial since, otherwise, it would be necessary to administer the compound to the entire group of patients for therapeutic and/or diagnostic (imaging) purposes and for some patients there would be no clinical benefit, e.g., a low benefit:risk ratio. Even at low doses of radiolabeled compound, e.g., ¹⁷⁷Lu-PP-F11N, the energy of the emitted radiation can be so strong that undesired side effects can easily occur. The selection of patients therefore significantly increases the efficacy of any kind of treatment and/or imaging, while minimizing side effects.

The compound targeting CCK2-R can be administered in any effective dose. In one embodiment, the compound targeting CCK2-R is a radiolabeled gastrin analogue of formula (1) wherein the radionuclide is ¹⁷⁷Lu, and the effective dose of the compound targeting CCK2-R administered to the patient is preferably selected from:
(i) a treatment dose of from 1.0 to 15.0 Giga-becquerels (GBq), preferably from 2.0 to 12.0 Gbq, more preferably from 5.0 to 10.0 GBq, in particular from 6.0 to 8.0 GBq, such as about 6.5 GBq; and
(ii) an imaging dose of from 0.5 to 3.0 GBq, preferably from 0.7 to 2.5 GBq, more preferably from 1.0 to 2.0 GBq, such as about 1.85 GBq.

In another embodiment, the compound targeting CCK2-R is a radiolabeled gastrin analogue of formula (1) wherein the radionuclide is ⁶⁸Ga, and the effective dose of the compound targeting CCK2-R administered to the patient is preferably (an imaging dose of) from 0.5 to 4 MBq/Kg/person, preferably from 1 to 3 MBq/Kg/person, such as about 2 MBq/Kg/person.

The compound can be administered to the patient at one time, or over a series of administration cycles e.g. in the case where the compound is a radiopharmaceutical such as a radiolabeled gastrin analogue of formula (1). In the context of imaging, the compound is preferably administered at one time, but repeated administration can also be useful for monitoring purposes, e.g., monitoring disease progression. The compound can be administered to the patient once or twice per cycle of two to ten weeks, preferably once per cycle of four to eight weeks, more preferably once per cycle of six weeks or once per cycle of eight weeks. When the compound is administered twice per cycle, the effective dose (treatment dose) is split in two half-doses which are administered separately over the course of the cycle. The number of cycles can range from one to a maximum of ten cycles, for instance one to eight or one to six cycles, e.g., three or four cycles.

In one aspect, the compound targeting CCK2-R is a radiopharmaceutical such as a radiolabeled gastrin analogue of formula (1), e.g., ¹⁷⁷Lu-PP-F11N, and the treatment and/or imaging can be performed by administering the compound according to one of the following administration patterns:
(A) once per cycle of six weeks for at least two six-week cycles, preferably once per cycle of six weeks for at least three six-week cycles, more preferably once per cycle of six weeks for three six-week cycles; or
(B) once per cycle of eight weeks for at least two eight-week cycles, preferably once per cycle of eight weeks for at least three eight-week cycles, more preferably once per cycle of eight weeks for three eight-week cycles.

According to another embodiment, the method of selectively treating and/or imaging cancer comprises the steps of:
(a) assaying a tumor sample from a patient diagnosed with cancer for the mRNA expression level of CCKBR;
(b) determining whether the mRNA expression level of CCKBR is equal to, or greater than a predetermined cut-off range to predict if the patient is likely to respond to treatment with a compound targeting CCK2-R;
(c) selectively administering an imaging dose of a first compound targeting CCK2-R to the patient to obtain an image of body parts or tissues; and
(d) selectively administering a treatment dose of a second compound targeting CCK2-R to the patient.

Wherein both the first and second compounds targeting CCK2-R are preferably radiopharmaceuticals (radiolabeled compounds), such as a radiolabeled gastrin analogue of formula (1), as disclosed herein.

In accordance with the second embodiment, the steps (a) and (b), and the features defined therein (i.e., tumor sample, mRNA expression level assay, patient diagnosis, cut-off range/value), are as defined above with respect to the method for predicting the response to treatment and/or imaging with a compound targeting CCK2-R, e.g., a radiolabeled gastrin analogue.

The first compound targeting CCK2-R can be any compound capable to bind to CCK2-R, such as a CCK2-R agonist, or a CCK2-R antagonist. In one embodiment, the first compound targeting CCK2-R is radiolabeled. Preferably, the first compound targeting CCK2-R (used in step (c)) is a radiolabeled gastrin analogue of formula (1) (as defined above), which is labeled with a first radionuclide.

The compound is selectively administered to a patient who has been identified in step (b) as CCK2-R, i.e., as likely to respond to treatment with a compound targeting CCK2-R, for imaging purposes. Hence, the mRNA expression level of CCKBR in the tumor sample is used in step (b) to (pre-)select the patients that are expected to benefit from treatment with the compound. Thereafter, the administration of a first compound targeting CCK2-R is used in step (c) for imaging purposes, i.e., to confirm CCK2-R positivity by visualization of the tumor tissues. Therefore, step (c) can be used to further select the patients that are expected to benefit from treatment with the compound. This selection process is very beneficial as it enables to identify the patients who are the most likely to benefit from treatment, thereby further increasing efficacy while minimizing side effects.

When the first compound targeting CCK2-R is a radiolabeled gastrin analogue of formula (1), the first radionuclide can be any radionuclide (or “tracer”) suitable for imaging body parts or tissues by standard imaging techniques, such as PET, Single Photon Emission Computed Tomography (SPECT), or the like. The first radionuclide can be selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm, preferably from ¹⁷⁷Lu, ⁹⁰Y, ⁶⁸Ga, ²²⁵Ac and ¹¹¹In, more preferably from ¹⁷⁷Lu and ⁶⁸Ga.

In one embodiment, the first radionuclide is ¹⁷⁷Lu, and the imaging dose of the first compound targeting CCK2-R administered to the patient is preferably from 0.5 to 3.0 GBq, preferably from 0.7 to 2.5 GBq, more preferably from 1.0 to 2.0 GBq, such as about 1.85 GBq.

Preferably, the imaging technique used to obtain an image in step (c) is PET. Visualization is achieved by recording the energy and location of the radiation emitted by the radionuclide, such as ⁶⁸Ga, this information then being used by a computer program to reconstruct three-dimensional (3D) images of radionuclide concentration within the body. In modern PET computed tomography scanners, PET images are often reconstructed with the aid of a computed tomography scan performed on the patient during or shortly after the administration of the tracer, in the same device.

PET images typically show a very high resolution, typically much higher than that achievable by SPECT, especially if they are obtained with e8Ga. SPECT is similar to PET in its use of radioactive tracer material. However, a PET scanner detects these emissions “coincident” in time, which provides more radiation event localization information and, thus, higher spatial resolution images than SPECT (which has about 1 cm resolution).

In a preferred embodiment, the first radionuclide is ⁶⁸Ga (and thus the first compound targeting CCK2-R is ⁶⁸Ga-PP-F11N), and the imaging dose administered to the patient is preferably from 0.5 to 4 MBq/Kg/person, more preferably from 1 to 3 MBq/Kg/person, such as about 2 MBq/Kg/person.

The convenient half-life of ⁶⁸Ga (T^{1/2}=68 min) provides sufficient radioactivity for various PET imaging applications. ⁶⁸Ga decays 87.94% through positron emission with a maximum energy of 1.9 MeV, mean 0.89 MeV. The ⁶⁸Ga³⁺ cation can form stable complexes with many ligands containing oxygen and nitrogen as donor atoms, particularly with DOTA.

In one embodiment, the method can comprise a step (after step (d)) of:
(e) administering an imaging dose of the first compound targeting CCK2-R, e.g., ¹⁷⁷Lu-PP-F11N or ⁶⁸Ga-PP-F11N, to the patient to obtain an image of body parts or tissues.

Step (e) can be used, e.g., to monitor the state of disease, to monitor the efficacy of the treatment with the second compound targeting CCK2-R, to adapt the dosage of second compound, or the like. It can be performed at one time, or after each administration (cycle) of the second compound targeting CCK2-R, if considered necessary.

The second compound targeting CCK2-R can be any compound capable to bind to CCK2-R, such as a CCK2-R, agonist or a CCK2-R antagonist. In one embodiment, the second compound targeting CCK2-R is radiolabeled. Preferably, the second compound targeting CCK2-R (used in step (d)) is a radiolabeled gastrin analogue of formula (1) (as defined above), which is labeled with a second radionuclide.

The compound is selectively administered to a patient who has been identified as CCK2-R positive, i.e., as likely to respond to treatment with a compound targeting CCK2-R, for treatment purposes. Therefore, the mRNA expression level of CCKBR in the tumor sample is used in step (b) to (pre-)select the patients that are expected to benefit from treatment and/or imaging with the compound, while imaging of the tumor tissues is performed in step (c) to further select the patients or confirm CCK2-R positivity. This is very beneficial as it enables to target the patients who are the most likely to respond to treatment with the second compound targeting CCK-2R, thereby increasing therapeutic efficacy while minimizing side effects.

When the second compound targeting CCK2-R is a radiolabeled gastrin analogue of formula (1), the second radionuclide can be selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm, preferably from ¹⁷⁷Lu, ⁹⁰Y, ⁶⁸Ga, ²²⁵Ac and ¹¹¹In, more preferably from ¹⁷⁷Lu and ⁶⁸Ga.

In one preferred embodiment, the second radionuclide is ¹⁷⁷Lu (and the second compound targeting CCK2-R is ¹⁷⁷Lu-PP-F11N), and the treatment dose administered to the patient is preferably from 1.0 to 15.0 GBq, more preferably from 2.0 to 12.0 Gbq, even more preferably from 5.0 to 10.0 GBq, in particular from 6.0 to 8.0 GBq, such as about 6.5 GBq.

In an even more preferred embodiment, the first radionuclide is ⁶⁸Ga, the imaging dose administered to the patient being preferably from 0.5 to 4 MBq/Kg/person, more preferably from 1 to 3 MBq/Kg/person, such as about 2 MBq/Kg/person, and the second radionuclide is ¹⁷⁷Lu, the treatment dose administered to the patient being preferably from 1.0 to 15.0 GBq, more preferably from 2.0 to 12.0 Gbq, even more preferably from 5.0 to 10.0 GBq, in particular from 6.0 to 8.0 GBq, such as about 6.5 GBq.

In yet another embodiment, the first and second radionuclides are identical, e.g., ¹⁷⁷Lu.

The second compound targeting CCK2-R can be administered to the patient at one time, or over a series of administration cycles. The compound can be administered to the patient once or twice per cycle of two to ten weeks, preferably once per cycle of four to eight weeks, more preferably once per cycle of six weeks or once per cycle of eight weeks. When the compound is administered twice per cycle, the effective dose (treatment dose) is split in two half-doses which are administered separately over the course of the cycle. The number of cycles can range from one to a maximum of ten cycles, for instance one to eight or one to six cycles, e.g., three or four cycles.

In one aspect, the second compound targeting CCK2-R, e.g., a radiolabeled gastrin analogue of formula (1), is administered according to one of the following administration patterns:
(A) once per cycle of six weeks for at least two six-week cycles, preferably once per cycle of six weeks for at least three six-week cycles, more preferably once per cycle of six weeks for three six-week cycles; or
(B) once per cycle of eight weeks for at least two eight-week cycles, preferably once per cycle of eight weeks for at least three eight-week cycles, more preferably once per cycle of eight weeks for three eight-week cycles.

According to the embodiments described above, the compound is administered to the patient by injection, in particular by intravenous injection. In this regard, the compound can be provided as a solution in a pharmaceutically acceptable injectable carrier such as an aqueous carrier (e.g., water or 0.9% sodium chloride). When the compound targeting CCK2-R is a radiolabeled compound, in particular a radiolabeled gastrin analogue of formula (1), the solution of the compound for injection can have a concentration of radiolabeled gastrin analogue ranging from 300 to 500 MBq/mL, such as about 400 MBq/mL. The infusion rate can be of from 35 to 60 mL/h, for instance about 50 mL/h.

In one embodiment, the above methods can comprise the steps of:
(α) preparing an injectable solution of the compound by dissolving the compound in a pharmaceutically acceptable injectable carrier to obtain an injectable solution of the compound, preferably an injectable solution having a concentration of the compound of 300 to 500 MBq/mL, e.g., 400 MBq/mL when the compound targeting CCK2-R is a radiolabeled gastrin analogue of formula (1); and
(β) administering the injectable aqueous solution of the compound obtained from step (a) to the patient, preferably at an infusion rate of 35 to 60 mL/h such as 50 mL/h over an infusion period of 20 to 60 min, e.g., 30 to 45 min when the first compound targeting CCK2-R is a radiolabeled gastrin analogue of formula (1).

In one embodiment, the compound is administered concurrently with, before and/or after one or more other therapeutic agents or therapies, such as DNA damage response (DDR) inhibitors, chemotherapeutic agents, immunomodulatory agents, proton pump inhibitors (PPIs), histamine H₂-receptor antagonists, tyrosine kinase inhibitors, or any other targeted therapies. In one further embodiment, the compound is administered concurrently with and/or before another therapeutic agent selected from a DDR inhibitor, a PPI, such as pantoprazole, and a histamine H₂-receptor antagonist, such as ranitidine, preferably concurrently with and/or before another therapeutic agent selected from pantoprazole and ranitidine, more preferably concurrently with and/or before pantoprazole.

### 5. Preparation of Radiolabeled Gastrin Analogues

In the following, methods are provided for the preparation of the radiolabeled gastrin analogue. The gastrin analogue can be synthesized relying on standard Fmoc-based solid-phase peptide synthesis (SPPS), including on-resin peptide coupling and convergent strategies. The general strategies and methodology which can be used for preparing and radiolabeling the gastrin analogue of the present invention are well-known to the skilled person and also described further below.

### 6. Examples

### 6.1 List of Abbreviations

BSA bovine serum albumin
DIEA: diisopropylethylamine
DMF: dimethyl formamide
DMSO: dimethyl sulfoxide
EGTA: ethylene glycol-bis(3-aminoethyl ether)-N,N,N′,N′-tetraacetic acid
ESI: electron spray ionization
HATU: 1-[Bis(dimethylamino)methylene]-1H-1,2,3-triazolo[4,5-b]pyridinium 3-oxide hexafluorophosphate
HBTU: 3-[Bis(dimethylamino)methyliumyl]-3H-benzotriazol-1-oxide hexafluorophosphate
HPLC: high-performance liquid chromatography
IU: international unit
PBS: phosphate-buffered saline
SQD: single quadrupole detection
SPECT: single-photon emission computed tomography
SPPS: solid-phase peptide synthesis
TBST: tris-buffered saline with Tween 20
TFA: trifluoroacetic acid
TIS: triisopropylsilane
Tris: tis(hydroxyethyl)aminomethane
UPLC: ultra-performance liquid chromatography

### 6.2 Materials and Methods

The following materials and methods were used to evaluate the compound and methods of the present invention.

### 6.2.1 Preparation of the Gastrin Analogue

The gastrin analogue described and used herein (PP-F11N) was prepared by standard Fmoc-based SPPS, including on-resin peptide coupling and convergent strategies using an Activo-P-11 Automated Peptide Synthesizer (Activotec) and a Rink Amide resin (loading: 0.60 mmol/g; Novabiochem).

Coupling reactions for amide bond formation were performed over 30 min at room temperature using 3 eq of Fmoc-amino-acids activated with HBTU (2.9 eq) in the presence of DIEA (6 eq). Fmoc deprotection was conducted with a solution of 20% piperidine in DMF. Coupling of the N-terminal labeling moiety can be performed over 30 min at room temperature using 3 eq of DOTA tris-t-Bu ester (Novabiochem) activated with HATU (2.9 eq) in the presence of DIEA (6 eq).

The peptide was cleaved from the resin under simultaneous side-chain deprotection by treatment with TFA/TIS/water (95/2.512.5, v/v/v) during 60 min. After concentration of the cleavage mixture, the crude peptide was precipitated with cold diethyl ether and centrifugated.

The peptide was purified on a Waters Autopurification HPLC system coupled to SQD mass spectrometer with a XSelect Peptide CSH C18 OBD Prep column (130 Å, 5 μm, 19 mm×150 mm) using solvent system (0.1% TFA in water) and B (0.1% TFA in 35 acetonitrile) at a flow rate of 25 mL/min and a 20-60% gradient of B over 30 min. The appropriate fractions were associated, concentrated and lyophilized. The purity was determined on a Waters Acquity UPLC System coupled to SQD mass spectrometer with CSH C18 column (130 Å, 1.7 μm, 2.1 mm×50 mm) using solvent system A (0.1% TFA in water) and (0.1% TFA in acetonitrile) at a flow rate of 0.6 mL/min and a 5-85% gradient of B over 5 min.

MS-analysis was performed using electrospray ionization (ESI) interface in positive and negative mode.

### 6.2.2 Radiolabeling of the Gastrin Analogue

### Lutetium Radiolabeling:

To prepare the lutetium-labeled gastrin analogue (¹⁷⁷Lu-PP-F11N; test compound), a solution of N-terminal DOTA-conjugated gastrin analogue PP-F11N (DOTA-(DGlu)₆-Ala-Tyr-Gly-Trp-Nle-Asp-Phe) prepared as described above and ¹⁷⁷Lu (available from ITG GmbH) in a nuclide/peptide ratio of 1:30 was prepared in 0.4 M ammonium acetate buffer (pH 5.5) and the labeling was carried out at 90° C. for 15 min.
The lutetium incorporation was analyzed by standard HPLC using a C18 column and reached above 95% efficiency.

### Indium Radiolabeling:

To prepare the indium-labeled gastrin analogue used in Example 1 below (¹¹¹In-PP-F11N), a solution of PP-F11N was added to the radionuclide solution (¹¹¹InCl₃ in 20 mM HCl, available from Curium). Labeling buffer (sodium acetate pH 5.3) was added to a final concentration of 0.1 M buffer. After heating for 25 min at 80° C., the reaction mixture was allowed to cool down for 5 min before adding 1 μl 10 mM DTPA and 1 μl 5% TWEEN-20 per 50 μl. For quality control, the reaction mixture was diluted 1:10 in HPLC sample diluent (0.1% TWEEN-20 in 0.1 M sodium acetate pH 5.3).
Labeling efficiency and radiochemical purity were determined by HPLC using an Agilent Poroshell HPH C18 column (gradient: 5% acetonitrile (ACN) to 70% ACN in 0.1% TFA in water within 15 min; flow rate: 0.5 ml/min). Labeling efficiency and radiochemical purity of ¹¹¹In-PPF11N was greater than 94%.

### Gallium Radiolabeling:

To prepare the Gallium-labelled gastrin analogue (⁶⁸Ga-PP-F11N; Imaging compound), an eluate of a gallium generator was added to a solution of PP-F11N 80 μg, Mannitol 6 mg, ascorbic acid 1 mg and sodium acetate (100Mg) (pH 3.9). After heating at 95° C. for 20 min, the reaction mixture was allowed to coal down for 10 min. The radiochemical purity was analyzed by TLC (thin layer chromatography) 5 μL sample on silica gel plate. Mobile phase 77 g/L solution of ammonium acetate in water, methanol 50:50 V/V. Detection with a detector suitable to determine the distribution of radioactivity. No more than 3% of free gallium-68

### 6.2.3 Tissue Acquisition and Preparation

Tissue (fresh frozen blocks) isolated from twenty SCLC, four twenty GC and twenty PDAC patients were acquired from a Tissue Biobank supplier. Tissues were allowed to equilibrate for at least 2 h in the cryotome chamber of a Leica 3050 before sectioning at −18° C. (chamber temperature) and at a thickness of 20 μm.

### 6.2.4 Autoradiography

The following buffers were prepared freshly before each assay.

TABLE 1
Autoradiography buffers

| | |
|---|---|
| Pre-incubation buffer (Pre-IB) | 0.05M Tris |
| | 130 mM NaCl |
| | 4.7 mM KCl |
| | 1 mM EGTA |
| | 0.5% BSA |
| | 5 mM MgCl₂ |
| | pH 7.4 |
| Incubation buffer (IB) | Pre-IB without BSA |
| | 0.025% Bacitracin |
| | 25 mg Dithiothreitol |
| | 2 μg/ml Chymostatin |
| | 4 μg/ml Leupeptin |
| | pH 6.5 |
| Wash buffer 1 (WB1) | Pre-IB with 0.5% BSA |
| | pH 7.4 |
| Wash buffer 2 (WB2) | Pre-IB without BSA |
| | pH 7.4 |

### Autoradiography Protocol:

The samples (tumor sections) were first dried for at least 5 min using a cold fan to increase tissue adsorption to the slides. Incubation with Pre-IB to reduce potential occupation of the CCKBR was followed by another drying step for 10 min. Afterwards the samples were incubated with ¹¹¹In-PPF11N in IB. In order to assess non-specific binding, an adjacent section was incubated in tracer solution mixed with 200 nM of unlabeled human gastrin I (available from Bachem, Switzerland). After the procedure, slides were washed 6×15 min in pre-cooled WB1 and 2×5 sec in WB2, before drying the sections for at least 15 min using a cold fan. Sections were apposed to a Biomax MR film in X-ray cassette and films were developed in an automated developing machine.

### Signal Quantification:

For signal quantification, a separate standard curve was recorded for every experiment. Autoradiograms were quantitatively analysed using MCID software (InterFocus). The region of interest (ROI) was measured twice, once on the tissue with total tracer binding and once on the sample for non-specific binding. Radioligand binding was considered specific if total binding signal was at least twice as high as the non-specific signal (Reubi et al. Cancer Res. 1997, 57(7), 1377-1386). Because the different tumor sample sections are composed of tumoral tissue and normal adjacent tissue, the colocalization of the radio-signal to the tumoral tissue in the slides was confirmed by a Hematoxylin and Eosin (H&E) staining.
   Hematoxylin and Eosin (H & E) staining:
H&E-staining of sections adjacent to the ones used in autoradiography permits localization of the autoradiographic signal. For H&E-staining, frozen tissue sections were fixated for 10 s in 1:1 acetone-ethanol solution (trichloroacetic acid 1 mol/l). Afterwards, they were hydrated in alcohol series (100; 96; 70; 50% EtOH) followed by a brief rinse in H₂O. Incubation in Mayer's hemalaun solution for 10 min stained the nuclei of cells. After washing in H₂O and dd H₂O, the slides were immersed briefly in hydrochloric acid alcohol. Subsequent 10 min incubation in warm water led to a colour change from red to blue. Counterstaining with eosin solution produces a contrast in acidophilic structures (pink). Slides were washed and dehydrated in an ascending alcohol series (70-100%). The samples were placed into xylene twice and subsequently embedded using Eukitt and glass cover slips.

### 6.2.5 Statistical Analysis

Statistical analysis is performed to assess the correlation between the correlation between specific binding of radiolabeled gastrin analogue (¹¹¹In-PP-F11N) to CCK2-R and the mRNA expression level of CCKBR.

Since the relationship between the percentage of specific binding (denoted Y) and the mRNA expression (denoted Z) is unlikely to be linear, the common Person correlation coefficient is not adequate. Then the correlation between both endpoints is assessed through a χ²-test (see Fisher, R. A. (1922), On the Interpretation of a χ² from Contingency Tables, and the Calculation of P. Journal of the Royal Statistical Society 1922, 85(1), 87-94). To perform the χ²-test, both endpoints, specific binding and mRNA expression level, are categorized as follows:
\tilde{Y}ᵢ=1{Yᵢ≥a}∀i=1 . . . X, with a being the cut-off value to define a high percentage of specific binding and X the number of analysed samples
\tilde{Z}ᵢ=1{Zᵢ≥b}∀i=1 . . . X, with b being the cut-off value to define a high mRNA expression level and X the number of analysed samples

The correlation between Y and Z is defined as the p-value, p(a,b), of the χ²-test between \tilde{Y} and \tilde{Z}.

The optimal cut-off values (a*, b*) are defined as follows:

$$( {a}^{*} , {b}^{*} ) = {arg min}_{( a , b )} p ( a , b )$$

Such optimal cut-off values maximize the consistency between categorized percentages of specific binding and mRNA expressions.

### Example 1: Association Between Specific Binding of Radiolabeled Gastrin Analogue (¹¹¹In-PP-FF11N) and mRNA Expression Level in Biological Samples

The objective of this example was to assess the correlation between the specific binding of radiolabeled gastrin analogue (¹¹¹In-PP-F11N) to CCK2-R and the mRNA expression level of CCKBR in tumor tissues and healthy tissues (the healthy tissues being used as positive/negative controls).

57 tissue samples were obtained and characterized for their ¹¹¹In-PP-F11N-binding and mRNA expression levels. The dataset combined samples collected from patients diagnosed with GIST, SCLC, NSCLC or MTC (i.e., 17 GIST samples, 1 GIST-PDX sample, 13 SCLC samples, 17 SCLC-PDX samples, 4 NSCLC samples, 2 MTC samples), as well as samples collected from healthy tissues, i.e., stomach, lung, kidney, to be used as positive (stomach) and negative (lung, kidney) controls. The specific binding of ¹¹¹In-PP-F11N in each sample was measured by autoradiography (as described above), while the mRNA expression level was measured by mRNA sequencing of the whole transcriptome. The mRNA expression level measurement was performed by Caris Life Sciences®.

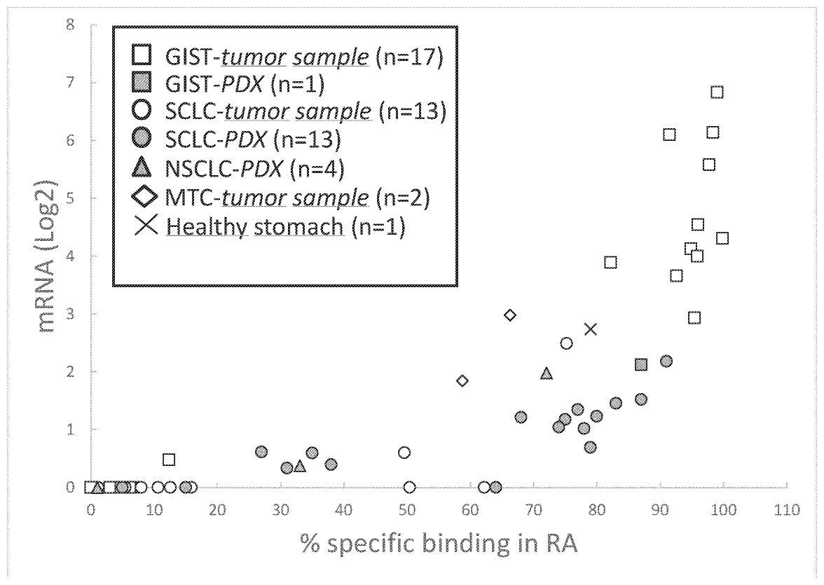

The correlation between specific binding of ¹¹¹In-PP-F11N to CCK2-R and mRNA expression level of CCKBR in the samples was analysed by χ² statistical analysis (as described above). The results are depicted in FIG. 1.

The statistical analysis showed that the relevant cut-off values to distinguish double negative from double positive samples is in the range of 50 to 65% for the specific binding of radiolabeled ligand and 0.6 to 1.0 for the log 2 of the mRNA expression level of CCKBR. The cut-off values determined for the specific binding are in accordance with the cut-off values reported in the literature for the same types of tumor tissues (see Reubi et al. Cancer Res 1997, 57(7), 1377-1386). Moreover, the related ratio of identical classification between autoradiography and mRNA sequencing measurements was found to be very high (from 93% to 96%). These results therefore demonstrate that the mRNA expression level of CCKBR constitutes a robust and reliable predictive biomarker of the clinical response of a particular patient to treatment and/or imaging with radioligands targeting CCK2-R.

### Example 2: Multicenter Phase 1a/1b Study of Safety, Tolerability, Whole-Body Radio-Distribution, and Radiation Dosimetry of ¹⁷⁷Lu-PP-FF11N in Patients with Unresectable Locally Advanced or Metastatic Solid Tumors, Selected by the Use of ⁶⁸Ga-PP-FF11N as Diagnostic Positron Emission Tomography Radio-Tracer

Overview of study design: This is a multicenter, multi-arm, open-label Phase 1a/1b study of safety, tolerability, pharmacokinetics (PK), pharmacodynamics (PDy), radiation dosimetry and preliminary antitumor activity of ¹⁷⁷Lu-PP-F11N (hereinafter the “test compound”), in patients with unresectable locally advanced or metastatic tumors.

The study consists in two parts, i.e., (1) Phase 1a, and (II) Phase 1b, as follows:
(I) Phase 1a: This part enrolls 6 patients with small cell lung cancer (SCLC) with unknown CCK2R expression status at the time of Study Screening. Upon confirmation of eligibility, all patients in Phase 1a are administered with the test compound at an activity dose of 1.85 GBq, used as imaging diagnostic tool to enable distribution and dosimetry estimations.
(II) Phase 1b: This part is initiated upon completion of Phase 1a and enrolls the following patient populations:
   Cohort 1: Up to 20 patients with SCLC
   Cohort 2: Up to 20 patients with gastrointestinal stromal tumors (GIST)
   Cohort 3: Up to 30 patients in a basket cohort consisting of:
      Up to 10 patients with Colorectal Cancer (CRC)
      Up to 10 patients with Non-Small Cell Lung Cancer (NSCLC)
      Up to 10 patients with Breast Cancer (BC)
   Cohort 4: Up to 10 patients with tumors of any histological type.

Before entering the Screening period, patients are pre-selected for CCK2-R expression, as follows:
For cohorts number 1, 2 and 3: all patients must be centrally pre-screened for CCKBR mRNA expression. For that purpose, either an archival or a freshly acquired biopsy is required.
For cohort 4: Patients with known CCKBR expression determined by a local validated method. CCKBR expression results must be made available for determination by the Sponsor of their adequacy and confirmation of patient eligibility for screening.

Only patients whose tumor is positive for CCK2-R expression proceed to the Screening Phase. At the Screening Phase patients are further selected based on ⁶⁸Ga-PP-F11N (hereinafter the “imaging compound”) positron emission tomography (PET) imaging. Only patients who have a centrally read positive imaging compound PET imaging scan are eligible for treatment with the test compound (please see below for the definition of a positive imaging compound PET scan).

Upon confirmation of eligibility, all patients in Phase 1b are administered with the test compound a treatment dose of 6.5 GBq, which constitutes a treatment cycle. A patient may receive a maximum of 3 treatment cycles. Treatment cycles are administered every 6 weeks (+2 weeks window allowed).

### Objectives:

(I) Phase 1a: The primary objectives of Phase 1a are to: 1) assess the radiation dosimetry and radio-distribution in tumor and critical organs following the administration of a single imaging dose (1.85 Gbq; 50 mCi) of the test compound in patients with SCLC, 2) extrapolate the organ/tissue absorbed doses for determination of the treatment dose that is administered in the Phase 1b, and 3) assess the safety and tolerability of a single imaging dose of the test compound administered intravenously.
(II) Phase 1b: The primary objectives of Phase 1b are to: 1) assess the radiation dosimetry and radio-distribution in tumor and critical organs following the administration of a single treatment dose of the test compound, and 2) assess the safety and tolerability of a treatment dose of the test compound administered intravenously.

### Study Population:

The study population includes adult patients with histologically confirmed unresectable locally advanced or metastatic solid tumors, for which no standard therapy is available, or patients who, in the opinion of the treating physician, are unlikely to tolerate or to benefit from the standard of care. The study population is defined by the inclusion and exclusion criteria described below. No protocol waivers are granted. Patients are allocated to the following cohorts:
(I) Phase 1a: A single cohort of up to 6 evaluable adult (≥18 years old) female and male patients with a diagnosis of SCLC.
(II) Phase 1b:
   Cohort 1: Up to 20 patients with SCLC
   Cohort 2: Up to 20 patients with gastrointestinal stromal tumors (GIST)
   Cohort 3: Up to 30 patients in a basket cohort consisting of:
      Up to 10 patients with Colorectal Cancer (CRC)
      Up to 10 patients with Non-Small Cell Lung Cancer (NSCLC)
      Up to 10 patients with Breast Cancer (BC)
   Cohort 4: Up to 10 patients with tumors of any histological type.

Upon confirmation of eligibility, patients are enrolled without exceeding the screening period. Eligible patients receive treatment with the test compound at the enrollment date or within the following 7 days if required by logistic reasons at sites. Assuming a 50% screening failure rate, approximately 160 patients are screened and undergo study-related screening assessments to allow the enrollment of ˜80 patients in the Phase 1b.

### Inclusion Criteria:

1) Age ≥18 years.
2) Signed and dated written informed consent by the patient prior to any study-specific procedure.
3) For patients in Phase 1a: Histologically or cytologically confirmed SCLC, with unknown CCK2-R tumor status.
4) For patients in Phase 1b:
   4.1) Unresectable locally advanced or metastatic cancer (i.e., SCLC, GIST, NSCLC, CRC, BC) with known CCKBR mRNA expression level, identified through central validated molecular assay. Patients with other tumor types with known CCKBR expression determined by a locally validated method might be eligible for enrollment in cohort 4. Results of the CCKBR expression test must be made available for determination by the Sponsor of their adequacy and confirmation of patient eligibility for screening.
   4.2) Approximately half of Target Tumor lesions (as per RECIST V1.1) showing positive imaging compound scan at screening.
5) For all study patients: Radiographically documented disease progression or recurrence after at least one systemic treatment regimen. Prior treatment with more than one line of chemotherapy, immunotherapy, or immuno-chemotherapy combo regimen, is allowed.
6) At least one non-irradiated, non-central nervous system (CNS), measurable target lesion as per RECIST V1.1 documented after the last anticancer therapy.
7) ECOG performance score 0-1.
8) For patients in Phase 1b: Life expectancy >6 months as per investigator's best judgement.
9) Adequate laboratory function defined as:
   Serum creatinine <1.5×ULN or estimated glomerular filtration rate (eGFR)>50 mL/min based on the Chronic Kidney Disease-Epidemiology Collaboration (CKD-EPI) equation.
   Absolute neutrophil count (ANC)≥1,500/μL.
   Platelets ≥100,000/μL.
   Hemoglobin ≥9 g/dL.
   White blood cells >2000/μL.
   AST and ALT≤3×ULN (or ≤5×ULN if liver metastasis).
   Total bilirubin ≤1.5×ULN (or ≤2.0×ULN is allowed if the direct bilirubin level is within normal ranges and the elevation is limited to indirect bilirubin).
10) Female must have a negative urine pregnancy test prior to first dose of study drug; or female patients must have an evidence of non-child-bearing potential by fulfilling one of the following criteria at screening:
   Post-menopausal defined as aged more than 50 years and amenorrheic for at least 12 months following cessation of all exogenous hormonal treatments.
   Women under 50 years old would be consider postmenopausal if they have been amenorrheic for 12 months or more following cessation of exogenous hormonal treatments and with luteinizing hormone (LH) and follicle-stimulating hormone (FSH) levels in the post-menopausal range for the institution.
   Documentation of irreversible surgical sterilization by hysterectomy, bilateral oophorectomy, or bilateral salpingectomy but not tubal ligation.
11) Patients with reproductive potential and sexually active must use adequate birth control methods, defined as:
   For females: hormonal contraceptives, intrauterine device or double barrier contraception, i.e., condom+diaphragm, condom or diaphragm+spermicidal gel or foam.
   For males: double barrier contraception, i.e., condom+diaphragm, condom or diaphragm+spermicidal gel or foam.

### Exclusion Criteria:

1) Pregnancy or breast-feeding.
2) Concomitant second malignancy under active treatment.
3) Ongoing toxicity >grade 1 NCI-CTCAE v5.0 before treatment start, related to prior anticancer treatment (except for grade 2 alopecia, or stable grade 2 sensory neuropathy).
4) Symptomatic central nervous system (CNS) disease requiring active medical management or local intervention. Asymptomatic patients with CNS metastases who received prior therapeutic cranial irradiation and are on stable, decreasing, or no steroids are eligible.
5) Peptic ulcer considered acute, obstructing, penetrating or bleeding.
6) Prior external beam radiation therapy to more than 25% of the bone marrow.
7) Prior administration of endo-radioligand therapy.
8) Current spontaneous urinary incontinence.
9) Known incompatibility with radiographic contrast agents due to severe allergic reaction, hypersensitivity, or renal insufficiency.
10) Uncontrolled diabetes, evidence of bleeding diathesis, uncontrolled psychiatric disorders, serious infections, or any other medical condition that might be aggravated by the treatment on evaluation.
11) Any of the following cardiac criteria:
   a. Mean resting QT interval corrected by Fridericia's formula (QTcF)>470 msec (females) or >450 msec (males) obtained from 3 electrocardiograms (ECGs).
   b. Any clinically relevant abnormality in rhythm, conduction or morphology of resting ECG (e.g. complete left bundle branch block, third degree heart block, second degree heart block, PR interval >250 msec).
   c. Any factors that increase the risk of QTc prolongation or risk of arrhythmic events (e.g. heart failure, hypokalemia, congenital long QT syndrome, family history of long QT syndrome or unexplained sudden death under 40 years of age in first degree relatives or any concomitant medication known to prolong the QT interval).
12) Major surgery (involving any organ within the cranium, chest, abdomen, pelvis, or joint replacement) within 12 weeks before enrollment.
13) Any condition that precludes a raised arms position for prolonged imaging purposes.
14) Evidence of penetration of ⁶⁸Ga-PET radiotracer in CNS (i.e., positive scan) during screening.

### Study Compounds:

The test compound (¹⁷⁷Lu-PP-F11N) is provided as pre-formulated ready-to-use solution by a centralized radiopharmacy/manufacturing facility. The solution for infusion is given as a slow intravenous administration at an infusion rate of about 50 mL/h over a period of 30-45 minutes. A saline solution is infused in parallel at the same infusion rate (not higher than 50 mL/h) to flush the tubing. More information can be found in the pharmacy manual. A dedicated pump or a flebo-infusion system for radiolabeled compounds is used. A source of lutetium 177 (¹⁷⁷Lu) is used to calibrate the SPECT scans;
The imaging compound (⁶⁸Ga-PP-F11N) is provided as a kit containing a precursor component, with the peptide-ligand to be chelated with the radionuclide, and preparation components, with the excipients and ancillaries to perform radiolabeling. The peptide-ligand precursor must be radio-labelled with ⁶⁸Ga at the site or at a central radio-pharmacy facility, according to the applicable local regulations, to generate the solution of imaging compound for injection. The radiolabeled diagnostic is manufactured, quality controlled, released and administered within the shelf-life to secure an administered activity dose within the optimal range (3 MBq/kg, to a maximum of 200 MBq). The dose of imaging compound is to be injected as an intravenous bolus.

### Dosing Scheme:

### (I) Test Compound (¹⁷⁷Lu-PP-F11N):

Phase 1a: An imaging dose of test compound (1.85 GBq (50 mCi)) is administered to all patients participating in the Phase 1a, to conduct the radio-distribution and dosimetry study and to map the CCK2-R expression in unselected SCLC. The administration occurs on Day 1 following patient's enrollment.
The Principal Investigator (PI) evaluates and decides if a patient participating in the Phase 1a could be eligible to receive treatment doses of test compound after completing the dosimetry study (i.e., after Day 28±2 days). If eligible, the patient is administered with up to 3 doses of test compound every 6 weeks (+2 weeks window allowed) and is followed similarly to Phase 1b patients (see below). Patients from the Phase 1a part of study who qualify for treatment access are not eligible to become part of the Phase 1b part of the study.
The activity dose to be administered to patients is calculated according to the estimated day and time of injection on the basis of the physical decay of the radio-ligand (half-life 6.65 days). Because some fluctuation on the effective injection time may occur, up to 10% variation in the actual injected activity could be accepted for both, the test compound is administered at imaging dose (diagnostic) and at treatment dose.
Phase 1b: All Phase 1b patients (with the exception of cohort 4) entering in the screening period must have a positive confirmation of CCKBR mRNA expression by means of a central molecular assay of a tumor biopsy sample. For cohort 4 only, patients with known CCKBR expression as determined by a local validated method. The results must be available for determination by the Sponsor of the adequacy of the results and confirmation of eligibility for screening.
Patients eligible for treatment receive up to 3 consecutive treatment doses (i.e., maximum of 3 treatment cycles), administered every 6 weeks (+2 weeks window allowed). Test compound treatment doses of ˜6.5 GBq are administered on Day 1 of each cycle. Up to 10% variation in the actual injected activity dose is acceptable.
For patients participating in the confirmatory dosimetry set, 3D-quantitative SPECT-CT is conducted following the first administration in cycle 1 only. Dose adjustments can be implemented for additional cycles using individual dosimetry data.
Co-medication: Applicable to patients receiving the test compound at treatment dose only. Following the administration of a treatment dose of the test compound and for the following 14 days, patients receive 40 mg of pantoprazole (or dose-equivalent proton-pump inhibitor [PPI]). In patients with contraindications to receive PPIs, histamine H₂-receptor antagonists (e.g., ranitidine) can be used.

### (II) Imaging Compound (⁶⁸Ga-PP-F11N):

During screening, all patients are administered with the ⁶⁸Ga-PET diagnostic imaging compound, at a dose of 3 MBq/kg (up to a maximum total dose of 200 MBq). To be eligible for treatment with the test compound, patients must have a positive imaging compound PET imaging scan, defined as:
Approximately 50% of selected target lesions (as per RECIST V1.1) (representative of disease burden) showing radiotracer uptake, and
Avid tumor lesions based on uptake greater than the liver, and
Selected tumor lesions representative of tumor burden (as per RECIST V1.1) showing concordant positive ⁶⁸Ga-PET and ¹⁸F-FDG-PET.

### Study Phases, Periods, and Overall Procedures:

(I) Phase 1a: SCLC patients with unknown CCKBR tumor status participate in this part of the study and receive a single test compound imaging dose (1.85 GBq [50 mCi]) for molecular imaging, dosimetry calculations, and PK analysis. All patients that received the test compound for imaging are evaluable for safety. Patients are evaluable for dosimetry if they received the study drug at imaging dose and have available SPECT imaging scans in at least 3 time point days, with no major protocol deviation that could jeopardize the interpretation of the clinical trial results (see statistical analysis plan [SAP]).

### Screening Period:

Screening assessments are completed within 28 days prior to the start of the dosimetry period.

### Dosimetry Period:

The test compound is administered on Day 1. The test compound imaging dose to be administered on Day 1 is ordered at least 3 weeks in advance (i.e. Day −21±2 days) of the scheduled day of administration. Dosimetry, radio-distribution, PK, and biomarker sampling are performed for up to 7 days following the test compound infusion.

### Dosimetry Imaging:

At defined time points following the end of infusion, during the 7 days post-injection, whole body 3D quantitative (Q) SPECT/CT (2 or 3 bed position) focused on disease location (including at least thorax and abdomen) is acquired on: Day 1 at 90 min (±30 min) and 6 h (±2h), Day 2 at 24 h (±4h), Day 3 at 48 h (±4h), and Day 7 (±1 day) post-infusion end. Following acquisition, all images are anonymized and transmitted (uploaded) into a secured environment together with the Imaging and Patient Data Sheet provided, as detailed in the Imaging Acquisition Manual. All Images are centrally evaluated. Additional information can be found in the Imaging Acquisition Manual.

### Pharmacokinetic Assessments: Radio-Ligand

Urine samples and blood samples from the contralateral arm of drug infusion are collected for PK assessment at defined time points on Day 1, and then once on each subsequent measurement day, systematically before planned SPECT/CT imaging. Aliquots of blood, plasma and urine samples collected at each time point are red with a gamma counter.
(a) Blood collection: pre-infusion (between −1h and infusion start), 5 min (±3 min) before the end of the infusion (=during the infusion) and then at 10 min (±5 min), 30 min (±10 min), 60 min (±15 min), 3 h (±0.5 h), 6 h (±1 h), 24 h (±1 h), and 48 h (±4 h) post end-of-infusion.
(b) Urine collection: one urine sample collected pre-infusion (between −1h and infusion start), and total urine collected during the following time intervals: 0 to 90 min, 90 min to 6 h and 6 to 24 h post-infusion start. At each time point, the total volume of urine and exact time of collection are recorded. The volume of urine is disposed of according to the appropriate rules for radio-biological waste material, as applicable.

### Pharmacokinetic Assessments: Peptide-Ligand

Aliquots of collected urine and plasma are stored in ultra-low freezer for metabolite profiling, to explore PK and urine excretion of intact peptide ligand and any eventual metabolites.

### Pharmacodynamic Biomarkers: Blood SCC-Related Biomarker Assessments:

Blood samples from the contralateral arm of drug infusion are collected on Day 1 (pre-infusion) to monitor tumor burden. For patients who receive the test compound at treatment (please see below), additional blood samples are collected during Cycle 1 (Day 7 and Day 28), Cycle 2 (Day 1 [pre-infusion]) and Cycle 3 (Day 1 [pre-infusion]), as applicable, and at end of treatment (EOT). Aliquots of collected plasma and/or serum are stored in ultra-low freezer for further analysis of PDy biomarkers.

### Predictive Biomarker Assessments:

Patients are requested to provide a pre-test compound liquid biopsy, and if available, a tumor sample (archival or fresh), for the purpose of analyzing the expression of CCK2-R to evaluate the possible correlation with radio-ligand tumor uptake. Circulating Tumor Cell (CTCs) counting is also performed and analyzed in relation to disease burden at baseline.
Patients who receive treatment doses (refer to sub-section below) also provide liquid biopsies at the end of each treatment cycle (i.e., on pre-dose on Day 1 of the next cycle and at end of treatment [EOT]) to re-evaluate the CTC count, and CCK2-R/biomarker expression in available biological samples.

### Clinical Follow-Up:

For all Phase 1a patients, a clinical visit is conducted 1-week post-infusion and includes a physical examination and a safety/clinical assessment. Patients are clinically monitored for safety for 4 weeks following test compound infusion. An end of study (EOS) visit is conducted 28 days (±2 days) after the infusion, or earlier if the patient initiates another treatment.

### Treatment Access for Patients Participating in the Phase 1a:

After completion of the imaging-based dosimetry recordings, patients in the Phase 1a part may be deemed eligible on a case-by-case basis to receive the test compound at a treatment dose. Before initiation of treatment with the test compound, patients are requested to consent with the treatment plan, risks, and protocol requirements by reading, understanding, and signing the Phase 1b inform consent form (ICF). Ongoing safety review is conducted by the safety review committee (SRC) and a decision to treat the patient is made by the investigator according to the patient's clinical condition and individual dosimetry data. The calculation of the radiation activity dose of test compound to be administered to each patient takes into account the individual dosimetry data for each given patient. Test compound treatment follows the same overall plan and schedule of assessments designed for the Phase 1b patients receiving treatment doses (see below).
Patients in the Phase-1a who may initiate treatment with the test compound, are not considered as enrolled in the Phase 1b set.

### End of Study Participation for Patients in Phase 1a:

The end of study participation for patients is defined as 28 days (±2 days) after the infusion of the imaging dose of test compound (EOS visit), or earlier if the subject initiates another treatment, whichever happens first.
For patients receiving treatment with the test compound, the last study visit is determined by the date the patient completes the EOS visit at the end of the treatment period (i.e., 6 weeks±2 days) after the infusion of the treatment dose of test compound, or earlier if the subject initiates another treatment, whichever happens first.
(II) Phase 1b: Phase 1b is initiated if ≤2 out of 6 patients in the Phase 1a part of the study present clinically relevant study treatment related safety events (i.e., NCI-CTCAE v5.0 grade ≥3 not recovering to baseline in 4 weeks from dosing).

To be eligible to participate in the Phase 1b part of the study, patients with solid tumors as described above must have prior confirmation of positive expression of CCKBR mRNA by central molecular assay (prescreening), except for patients in cohort 4. For cohort 4 only, patients must have a known CCKBR mRNA expression status by local validated test and Sponsor's confirmation of eligibility based on those results.

Potentially eligible patients require to consent to prescreening activities (as described in the prescreening ICF). Either an archival or fresh biopsy is required for the prescreening. If the CCKBR mRNA expression level is confirmed to be above the defined cutoff level, the patient is offered to participate in the study and undergo the screening procedures upon informed consent signature. During screening, all patients are administered the imaging compound at a dose of 3 MBq/kg, up to maximum of a total administered activity of 200 MBq. Patients with a positive imaging compound PET scan are considered eligible to receive study treatment with the test compound provided all the remaining inclusion/exclusion criteria are satisfied.

A positive imaging compound PET scan is defined as:
Approximately 50% of selected target lesions (as per RECIST V1.1) (representative of disease burden) showing radiotracer uptake, and
Avid tumor lesions based on uptake greater than the liver, and
Selected tumor lesions showing concordant positive ⁶⁸Ga-PET and ¹⁸F-FDG-PET.

Eligible patients receive treatment with the test compound at the administered activity dose of 6.5 GBq per treatment cycle. Treatment dose adjustments can be implemented for cycles 2 and 3 based on individual dosimetry data. Each cycle consists of a single administration of test compound. Each study patient receives up to 3 cycles administered 6 weeks apart (+2 weeks window allowed).

The 6 first consecutive patients per tumor type, receiving the first dose of treatment with the test compound enter in the dosimetry set population. If more than 6 patients diagnosed with the same histological tumor type are enrolled into the trial, no additional dosimetry is conducted.

All patients receiving the imaging compound dose are evaluable for safety, regardless of whether the patient received treatment doses of test compound. Patients are considered evaluable for anti-tumor activity if they received at least one test compound treatment dose and have a baseline and post-baseline imaging-based tumor assessment with target lesions as per RECIST V1.1. To be evaluable for dosimetry, patients in the Phase 1b part must have available SPECT/CT scans on 3 different time points, at least. Patients who are not evaluable for dosimetry are not replaced.

### Screening Period:

Patients undergo screening assessments within 28 days prior to the initial test compound treatment dose. All patients are administered with the imaging compound at a dose of 3 MBq/kg, up to a maximum total dose of 200 MBq). Following the administration of the imaging compound, all patients are followed for safety (i.e., TEAEs) to the end of the screening period. Patients resulting in screen failure following imaging compound ⁶⁸Ga-PET scan are evaluated one week later as part of the clinical follow-up. ¹⁸FDG-PET procedure is conducted during this period. A CCK2-R-positive scan in approximately 50% of selected target lesions (as per RECIST V1.1) is required for the eligibility to receive the test compound treatment dose.
The test compound treatment dose is ordered once positive imaging compound PET imaging scan is confirmed.

### Treatment Period:

### Test Compound Treatment Doses:

Patients qualifying for treatment with the test compound are administered up to 3 doses of up to 6.5 GBq each, delivered in 3 consecutives treatment cycles separated by 6 weeks interval (plus up to 2 weeks window allowance). Currently available human radio-distribution and dosimetry data in healthy organs and tissues supports the proposed treatment plan with the test compound, as it shows that total cumulative radiation absorbed dose in critical organs is below the recommended threshold for the most sensitive ones.
Following the first dose of test compound, dosimetry assessments are conducted during the first week of treatment in patients enrolled in the dosimetry set population.
The first test compound treatment dose is administered on Day 1. In the absence of clinically relevant related safety events (i.e., NCI-CTCAE v5.0 grade 23 not recovering to baseline in 4 weeks from dosing), patients may receive up to 2 additional treatment doses of test compound (i.e., up to 3 treatment doses in total), every 6 weeks (plus up to 2 weeks window allowance).
Following administration of the test compound on the first day of each treatment cycle (Day 1), patients receive 40 mg of pantoprazole (or dose-equivalent PPI) daily for 14 days. In patients with contraindications to receive PPIs, histamine H₂-receptor antagonists (e.g., ranitidine) may be used.
In the presence of clinically relevant related toxicity (i.e., NCI-CTCAE v5.0 grade ≥3) not recovering to baseline in 4 weeks from the previous dose, the patient receives additional doses (no intra-patient dose reductions are allowed). If 2 or more patients in the Phase 1b part of the study cannot receive treatment doses due to unacceptable toxicity as described above, a 30% reduction of the administered dose is implemented for any potential new patient to enter the study.
Patients are clinically monitored for safety until the EOT visit (i.e., 6 weeks after the last dose of test compound). Full clinical and laboratory assessments are performed, as well as the estimation of the accumulated absorbed radiation dose in tumor and healthy organs.

### Confirmatory Dosimetry Set:

Confirmatory dosimetry is performed in patients enrolled in Phase 1b following the first treatment dose only. The first 6 consecutive patients per tumor type who receive treatment with the test compound in cycle 1 enter in the dosimetry set population. For the confirmatory dosimetry, the technical imaging modality consists of whole-body 3D Q-SPECT/CT covering from skull to the mid-thigh to ensure capturing signals from tumor and healthy organs. All the associated procedures are identical to those in the Phase 1a part of the study.
From the end of infusion of test compound, whole-body 3D Q-SPECT/CT (3 bed positions) images are acquired on Day 1 at 6 h (±2 h), on Day 2 at 24 h (±4 h), on Day 3 at 48 h (±4 h), and on Day 7 (±1 day) post-infusion.
Patients receiving treatment with the test compound but not entering in the dosimetry set population have a whole-body 3D Q-SPECT/CT scan on Day 2 at 24 h (±4 h) post-infusion, only. Following acquisition, all images (including those captured for the confirmatory dosimetry set) are anonymized and transmitted (uploaded) into a secured environment, as detailed in the imaging manual. All Images are centrally evaluated. Additional information can be found in the Imaging Manual.

### Tumor Imaging:

Standard morphological imaging: During treatment with the test compound, tumor assessments (as per RECIST v 1.1.) are conducted every 6 weeks, before the patient receives a new dose of test compound. After completion of the treatment (follow-up period) assessments are done every 12 weeks. All assessments should be done with the same image modality (computer tomography (CT)/magnetic resonance imaging (MRI)) used during baseline assessment.
Molecular imaging: For patients participating in the Phase 1a, SPECT/CT images are acquired at the following time points: Day 1 at 90 min (±30 min) and 6 h (±2h); Day 2 at 24 h (±4h); Day 3 at 48 h (±4h); Day 7 (±1 day) post end of infusion.
All patients participating in the Phase 1b part of the study are injected with the imaging compound as part of the screening. To become eligible for treatment with the test compound, patients must have a positive imaging compound PET imaging scan. Anonymized images are transmitted (uploaded) into a secured environment, as detailed in the imaging manual. All Images are centrally evaluated. Additional information can be found in the Imaging Manual.

### Pharmacokinetic Assessments: Radio-Ligand

Blood samples, taken from the contralateral arm where the infusion line for the test compound is placed, are collected for PK assessments at the following time points (before planned SPECT imaging):
   Cycle 1: pre-infusion (between −1 h and infusion start), 5 min (±3 min) before the end of the infusion (=during the infusion), and then at 10 min (±5 min), 30 min (±10 min), 60 min (±15 min), 3 h (±0.5 h), 6 h (±1 h), 24 h (±1 h) and 48h (±4 h) post end-of-infusion.
   Cycle 2 and 3: 5 min (±3 min) before the end of the infusion (=during the infusion), and 20 min (±10 min) and 6 h (±1 h) post end-of-infusion.
Plasma samples and urine from at least 6 patients, are collected during Cycle 1 for PK assessments at the following timepoints:
   Plasma collection: pre-infusion (between −1h and infusion start), 5 min (±3 min) before the end of the infusion (=during the infusion), and then at 10 min (±5 min), 30 min (±10 min), 60 min (±15 min), 3 h (±0.5 h), 6 h (±1 h), 24 h (±1 h) and 48h post end-of-infusion.
   Urine collection: one sample pre-infusion (between −1h and infusion start), and total urine collected during the following time intervals: 0 to 90 min, 90 min to 6 h and 6 to 24 h post infusion start.
Aliquots of blood, plasma and urine samples collected at each time point are read with a calibrated gamma counter.

### Pharmacokinetic Assessments: Peptide-Ligand:

Aliquots of collected urine and plasma are stored in ultra-low freezer (i.e., ≤−65° C.) for metabolite profiling, to explore PK and urine excretion of intact peptide ligand and any eventual metabolites.

### Pharmacodynamic Biomarkers: Blood SCC-Related Biomarker Assessment

For patients included in Cohort 1 (SCLC) blood samples from the contralateral arm of drug infusion are collected on Cycle 1 Day 1 (pre-infusion), during the Cycle 1 (Day 7 and Day 28), Cycle 2 (Day 1 [pre-infusion]) and Cycle 3 (Day 1 [pre-infusion]), as applicable, and at EOT. Aliquots of collected plasma and/or serum are stored in ultra-low freezer for further analysis of PDy biomarkers.

### Predictive Biomarker Studies

Patients are requested to provide a pre-test compound blood sample. Tumor Mutational Burden (TMB) and other biomarkers are also assessed in the blood sample.
Patients are asked to undergo an optional biopsy procedure. The newly acquired tumor biopsy is utilized for analysis of mRNA expression of relevant genes, including but not limited to CCKBR and its potential correlation with radio-ligand tumor uptake. The acquisition of the biopsy is not mandatory and patients who do not consent to it are not precluded from participation in the study.
Patients are also requested to provide blood samples pre-dose at the beginning of each treatment cycle (i.e., pre-dose on Day 1 of each cycle) and at EOT to explore a potential correlation between test compound anti-tumor activity, TMB and other biomarkers.

### Treatment:

All patients participating to the Phase 1b are followed up with regular visits after the infusion of the treatment agent, which includes a physical examination and safety/clinical assessments. Patients participating in the Confirmatory Dosimetry set in addition follow a specific imaging and biological sampling plan during the first week following treatment administration.
Six weeks after the last treatment dose, the EOT visit is performed, including disease assessment using the same imaging modality and procedure conditions as at baseline. The EOT visit should in any case be performed before any new antitumor therapy is given, should the patient be required to initiate another therapy.
All patients are evaluated for safety and tolerability based on the physical examination, vital signs, ECG, laboratory assessments including hematology, blood chemistry and urinalysis. These tests are performed according to the schedule of assessments. Safety is graded according to the NCI-CTCAE v5.0. Particular attention is paid to potential radiation-sensitive critical organs including the kidney, bone marrow, and abdominal viscera.

### Follow-Up:

After the EOT visit, all patients are followed for efficacy and safety with regular visits every 3 months until disease progression, initiation of new anticancer treatment, death or 6 months from the EOT visit of the last patient on study, whichever occurs first. Imaging-based tumor assessment should be performed the week before the FU visit.

### EOS for Phase 1b:

The EOS at an individual patient level occurs upon disease progression, death or 6 months from the EOT visit of the last on-study patient, whichever occurs first. Except in case of death, the patient undergoes the EOS assessments.
The global EOS is triggered once the last on-study patient reaches his/her 6-months post EOT visit. At this timepoint, all patients who have not previously discontinued from the study prematurely undergo the EOS assessments and are considered as having completed the study.

### Statistical Methods:

### Screening Period:

As data is reported descriptively, no formal statistical sample size calculation is required:
   Phase 1a: 6 evaluable patients. An evaluable patient is defined as a patient who received study drug administration at imaging dose and has available SPECT imaging scans on at least 3 timepoints on different days. Additionally, the patient must not have any major protocol deviation that could jeopardize the interpretation of the clinical trial results.
   Phase 1b: Up to 20 evaluable patients with SCLC, up to 20 evaluable patients with GIST, up to 30 evaluable patients entering in the basket cohort (CRC, BC, and NSCLC), and up to 10 evaluable patients with other tumor types enrolled in cohort 4. An evaluable patient is defined as a patient who received study drug administration at treatment dose and has a baseline and post-baseline imaging-based tumor assessment, with no major protocol deviation. Patients evaluable for dosimetry must have available SPECT/CT scans on at least 3 timepoints, with no major protocol deviation that could jeopardize the interpretation of the clinical trial results.
   It is anticipated that up to 160 patients may be screened in order to enroll approximately 80 evaluable patients into the Phase 1b.

### Timing of Analysis:

The following analyses are planned:
1. When all patients enrolled in Phase 1a have completed the participation in this study part, EOT visit or have discontinued from the study.
2. When all patients in the Phase 1b confirmatory dosimetry set have completed their dosimetry assessments.
3. For the Phase 1b cohorts:
   1. Cohort 1: When 6 patients have completed the EOT visit
   2. Cohort 2: When 6 patients have completed the EOT visit
   3. Cohort 3: When 6 patients per tumor type have completed the EOT visit
   4. Cohort 4: When 10 patients have completed the EOT visit
4. The final analysis takes place when all patients enrolled into the study have completed their EOS visit or have discontinued from the study.

### Safety Analysis:

The following analyses are planned:
TEAEs are graded using NCI-CTCAE v5.0. TEAEs, related TEAEs, TEAEs by severity, TEAEs leading to treatment discontinuation/modification, and serious adverse events (SAEs) are tabulated and listed by MedDRA system organ class and preferred term. Any on-study deaths and reasons are listed. Safety laboratory parameters and changes from baseline are presented as descriptive statistics by Phase and by timepoint. Shift tables are presented.

### Efficacy Analysis:

Disease assessment and anti-tumor activity based on RECIST v1.1 are presented by descriptive statistics for all evaluable patients.

### Other Analysis:

Comprehensive dosimetry analysis based on patients in Phase 1a administered with a test compound imaging dose, and patients with SCLC included in the confirmatory dosimetry set in Phase 1b.
Additional dosimetry analysis from patients with tumors other than SCLC, segregated by tumor type.
Pooled dosimetry data from normal healthy organs from all patients enrolled in the study.
Estimation of the recommended treatment dose for Phase 1b Cohort 1 (SCLC) based on the pooled tumor dosimetry data (absorbed dose) from Phase 1a.

### Safety Oversight:

During the conduct of Phase-1a part of study, an SRC evaluates the safety and tolerability of test compound infusions (diagnostic and treatment if applicable) in all patients individually. The SRC is composed of the participating investigators, including clinical oncologists, nuclear medicine physicians, and the Sponsor's medical director, pharmacovigilance (PV) representative, and biostatistician. If additional expertise is needed, appropriate supportive staff can be invited for the meetings. The roles and responsibilities of the SRC, as well as details about meeting format and frequency are defined in the SRC charter.

During the conduct of Phase-1b part of study, the SRC meets every 3 months to analyze the group of patients who received the imaging compound and the test compound, and make recommendations, if deemed necessary.

1-28. (canceled)

29. A method for predicting a response of a patient diagnosed with cancer to treatment and/or imaging with a compound targeting cholecystokinin 2 receptor, the method comprising the steps of:
(a) assaying a tumor sample from a patient diagnosed with cancer for the mRNA expression level of cholecystokinin B receptor; and
(b) determining whether the mRNA expression level of CCKBR is equal to, or greater than a predetermined cut-off range to predict whether or not the patient is likely to respond to treatment and/or imaging with a compound targeting CCK2R;
wherein the compound targeting CCK2R is a radiolabeled gastrin analog represented by formula:
X-DGlu-DGlu-DGlu-DGlu-DGlu-DGlu-Ala-Tyr-Gly-Trp-NIe-Asp-Phe-NH₂,
wherein X represents a moiety that chelates a radionuclide.

30. The method according to claim 29, wherein the moiety that chelates the radionuclide is DOTA or NODAGA.

31. The method according to claim 29, wherein the compound targeting CCK2R is a gastrin analog represented by formula, and the radionuclide is selected from the group consisting of ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, ^{99m}Tc, ²¹²Pb, ²¹²Bi, ²¹³Bi, ²¹¹At, ²⁵⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm.

32. The method according to claim 29, wherein the patient is diagnosed with a disease selected from medullary thyroid cancer, gliomas, small-cell lung cancer, extrapulmonary small-cell carcinoma, gastroenteropancreatic neuroendocrine tumors, gastrointestinal stromal tumors, non-small cell lung cancer, colorectal cancer, astrocytomas, stomach cancer, ovarian cancer, breast cancer, and any other disease expressing the cholecystokinin 2 receptor.

33. The method according to claim 29, wherein the patient is diagnosed with a disease selected from SCLC, GIST, CRC, BC and NSCLC.

34. The method according to claim 29, wherein the tumor sample is a biopsy sample selected from a paraffin-embedded and fixed biopsy sample, a fresh biopsy sample, a frozen biopsy sample, or a sample derived from a core needle or fine-needle aspiration biopsy.

35. The method according to claim 29, which comprises determining the mRNA expression level of CCKBR by reverse transcription-polymerase chain reaction, RNA sequencing, or any other method for assaying mRNA expression levels in cells.

36. The method according to claim 29, which comprises selecting the predetermined cut-off range from 0.4 to 2.0 log 2 transcripts per million, from 0.5 to 1.8 log 2 TPM, from 0.6 to 1.4 log 2 TPM, or from 0.6 to 1.0 log 2 TPM.

37. A compound for use in a method of selectively treating and/or imaging cancer in a patient diagnosed with the cancer, wherein:
the method comprises the following steps:
(a) assaying a tumor sample from the patient diagnosed with cancer for an mRNA expression level of CCKBR;
(b) determining whether the mRNA expression level of CCKBR is equal to, or greater than a predetermined cut-off range to predict whether or not the patient is likely to respond to treatment and/or imaging with a compound targeting CCK2R; and
(c) selectively administering an effective dose of compound targeting CCK2R to the patient;
the compound targeting CCK2R is a radiolabeled gastrin analog represented by the following formula:
X-DGlu-DGlu-DGlu-DGlu-DGlu-DGlu-Ala-Tyr-Gly-Trp-Nle-Asp-Phe-NH₂,
wherein X represents a moiety that chelates a radionuclide.

38. The compound according to claim 37, wherein the moiety that chelates the radionuclide is DOTA or NODAGA.

39. The compound for use according to claim 37, wherein the compound targeting CCK2R is a gastrin analog represented by formula, and the radionuclide is selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁷Ga, ^{99m}Tc, ²¹²Pb, ²¹²Bi, ²¹³Bi, ²¹¹At, ²²⁵Ac, ²²³Ra, ¹⁴⁹Tb, ¹⁶¹Tb, ²²⁶Th, ²²⁷Th, ⁸⁹Sr, ^{44/43}Sc, ⁴⁷Sc and ¹⁵³Sm.

40. The compound for use according to claim 37, wherein the patient is diagnosed with a disease selected from MTC, gliomas, SCLC, EPSCC, GEP-NET, GIST, NSCLC, CRC, astrocytomas, stomach cancer, ovarian cancer, BC, and any other disease expressing CCK2R.

41. The compound for use according to claim 37, wherein the patient is diagnosed with a disease selected from SCLC, GIST, CRC, BC and NSCLC.

42. The compound for use according to claim 37, wherein the tumor sample is a biopsy sample selected from a paraffin-embedded and fixed biopsy sample, a fresh biopsy sample, a frozen biopsy sample, or a sample derived from a core needle or fine-needle aspiration biopsy.

43. The compound for use according to claim 37, wherein the mRNA expression level of CCKBR is determined by RT-PCR, RNA sequencing, or any other method for assaying mRNA expression levels in cells.

44. The compound for use according to claim 37, wherein the predetermined cutoff range is selected from 0.4 to 2.0 log 2 transcripts per million, from 0.5 to 1.8 log 2 TPM, from 0.6 to 1.4 log 2 TPM, or from 0.6 to 1.0 log 2 TPM.

45. The compound for use according to claim 37, wherein the compound targeting CCK2R is a radiolabeled gastrin analog represented by formula wherein X chelates ¹⁷⁷Lu, and the effective dose of the compound administered to the patient is selected from:
(i) a treatment dose of between 1.0 and 15.0 GBq, or between 2.0 and 12.0 GBq, or between 5.0 and 10.0 GBq, or between 6.0 and 8.0 GBq, or about 6.5 GBq; and
(ii) an imaging dose of between 0.5 and 3.0 GBq, or between 0.7 and 2.5 GBq, or between 1.0 and 2.0 GBq, or about 1.85 GBq.

46. The compound for use according to claim 37, wherein the compound targeting CCK2R is a radiolabeled gastrin analog represented by formula wherein X chelates ⁶⁸Ga, and the effective dose of the compound administered to the patient is from 0.5 to 4 MBq/kg/person, or from 1 to 3 MBq/kg/person, or about 2 MBq/kg/person.

47. The compound for use according to claim 37, wherein the compound is administered to the patient once or twice per cycle of two to ten weeks, or once per cycle of four to eight weeks, or once per cycle of six weeks, or once per cycle of eight weeks.

48. The compound for use according to claim 37, wherein the compound is administered to the patient:
(A) once per cycle of six weeks for at least two six-week cycles, or once per cycle of six weeks for at least three six-week cycles, or once per cycle of six weeks for three six-week cycles; or
(B) once per cycle of eight weeks for at least two eight-week cycles, or once per cycle of eight weeks for at least three eight-week cycles, or once per cycle of eight weeks for three eight-week cycles.

49. The compound for use according to claim 37, wherein the compound is administered concurrently with, before, and/or after one or more other therapeutic agents or therapies, including DNA damage response inhibitors, chemotherapeutic agents, immunomodulatory agents, proton pump inhibitors, histamine H2-receptor antagonists, tyrosine kinase inhibitors, or any other targeted therapy.

50. A compound for use in a method of selectively treating cancer in a patient diagnosed with cancer, the method comprising the following steps:
(a) assaying a tumor sample from the patient diagnosed with cancer for the mRNA expression level of CCKBR;
(b) determining whether the mRNA expression level of CCKBR is equal to, or greater than a predetermined cut-off range to predict whether or not the patient is likely to respond to treatment with a compound targeting CCK2R;
(c) selectively administering an imaging dose of a first compound targeting CCK2R to the patient to obtain an image of body parts or tissues; and
(d) selectively administering a treatment dose of a second compound targeting CCK2R to the patient;
wherein the first compound targeting CCK2R is a radiolabeled gastrin analog represented by the following formula:
X-DGlu-DGlu-DGlu-DGlu-DGlu-DGlu-Ala-Tyr-Gly-Trp-Nle-Asp-Phe-NH₂, wherein X represents a moiety that chelates a first radionuclide; and
wherein the second compound targeting CCK2R is a radiolabeled gastrin analog represented by formula, wherein X represents a moiety that chelates a second radionuclide.

51. The compound according to claim 50, wherein the moiety that chelates the first radionuclide is DOTA or NODAGA and the moiety that chelates the second radionuclide is DOTA or NODAGA.

52. The compound according to claim 50, wherein each of the first and second compounds targeting CCK2R is a radiolabeled gastrin analog represented by formula, and wherein each of the first and second radionuclides is independently selected from ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ⁶⁸Ga, 99m_{TCj} 212p_{bj} 212_{Bii} 213\hat{ } 211_{At} 225\hat{ } 223_{Ra} 149_{Tbj} 161_{Tb} 226_{Th} 227_{Th} 89_{S I} 44/43\hat{ } ⁴⁷Sc and ¹⁵³Sm.

53. The compound according to claim 50, wherein each of the first and second compounds targeting CCK2R is a radiolabeled gastrin analog represented by formula, and wherein:
the first radionuclide is ⁶⁸Ga, and the imaging dose administered to the patient is from 0.5 to 4 MBq/kg/person, or from 1 to 3 MBq/kg/person, or about 2 MBq/kg/person; and/or
the second radionuclide is ¹⁷⁷Lu, and the treatment dose administered to the patient is from 1.0 to 15.0 GBq, or from 2.0 to 12.0 GBq, or from 5.0 to 10.0 GBq, or from 6.0 to 8.0 GBq, or about 6.5 GBq.

54. The compound according to claim 50, wherein the patient is diagnosed with a disease selected from the group consisting of MTC, gliomas, SCLC, EPSCC, GEP-NET, GIST, NSCLC, CRC, astrocytomas, stomach cancer, ovarian cancer, BC, and any other disease expressing CCK2R.

55. The compound according to claim 50, wherein the patient is diagnosed with a disease selected from the group consisting of SCLC, GIST, CRC, BC and NSCLC.

56. The compound according to claim 50, wherein the tumor sample is a biopsy sample selected from a paraffin-embedded and fixed biopsy sample, a fresh biopsy sample, a frozen biopsy sample, or a sample derived from a core needle or fine-needle aspiration biopsy.

57. The compound according to claim 50, wherein the mRNA expression level of CCKBR is determined by RT-PCR, RNA sequencing, or any other method for assaying mRNA expression levels in cells.

58. The compound according to claim 50, wherein the predetermined cut-off range is 0.4 to 2.0 log 2 transcripts per million, or 0.5 to 1.8 log 2 TPM, or 0.6 to 1.4 log 2 TPM, or 0.6 to 1.0 log 2 TPM.

59. The compound according to claim 50, wherein at least one of the first or second compound is/are administered concurrently with, before, and/or after one or more other therapeutic agents or therapies selected from DNA damage response inhibitors, chemotherapeutic agents, immunomodulatory agents, proton pump inhibitors, histamine-receptor antagonists, tyrosine kinase inhibitors, or any other targeted therapy.
